(19) United States
(12) Patent Application Publication
(10) Pub. No.: US 2025/0179158 A1
(43) Pub. Date: Jun. 5, 2025

(54) COMPOSITIONS OF ANTI-C5 MONOCLONAL ANTIBODY

(71) Applicant: AMGEN INC., Thousand Oaks, CA (US)

(72) Inventors: Anna IP, Moorpark, CA (US); Wenzhou LI, Thousand Oaks, CA (US); Cheryl Ann SAPHOS, Thousand Oaks, CA (US); Tian WANG, Newbury Park, CA (US); Dong XIANG, Thousand Oaks, CA (US); Jing YAN, Thousand Oaks, CA (US); Nathan JOH, Thousand Oaks, CA (US); Marissa JOUBERT, Newbury Park, CA (US); Siddharth PRABHU, Los Angeles, CA (US); Joshua TOKUDA, Cerritos, CA (US); Ahmed ELBARADEI, Oxnard, CA (US); Jette WYPYCH, Calabasas, CA (US)

(21) Appl. No.: 18/843,084

(22) PCT Filed: Mar. 1, 2023
(86) PCT No.: PCT/US2023/063460

§ 371 Date: Aug. 30, 2024

Related U.S. Application Data
(60) Provisional application No. 63/315,937, filed on Mar. 2, 2022.
(60) Provisional application No. 63/486,410, filed on Feb. 22, 2023.

(51) Int. Cl. C07K 16/18; A61K 39/00

(52) U.S. Cl. CPC C07K 16/18; A61K 2039/505; C07K 2317/24

(57) ABSTRACT

Pharmaceutical compositions comprising anti-C5 antibody are described herein. Methods comprising administering pharmaceutical compositions are described herein. Methods of manufacturing pharmaceutical compositions are described herein.

### REFERENCE TO RELATED APPLICATIONS

The present application claims the benefit of U.S. Provisional Application No. 63/315,937, filed Mar. 2, 2022, and U.S. Provisional Application No. 63/486,410, filed Feb. 22, 2023, each of which is hereby incorporated by reference in its entirety.

### SEQUENCE LISTING

The present application is being filed along with a Sequence Listing in electronic format. The Sequence Listing is provided as a file entitled 10111-WO01-SEC-SEQLISTING.TXT, created Feb. 27, 2023, which is 23.6 kilobytes in size. The information in the electronic format of the Sequence Listing is incorporated herein by reference in its entirety.

### FIELD

The instant disclosure relates to molecular attributes of an antibody and compositions thereof.

### BACKGROUND

The complement system comprises a series of proteins that interact with one another in a cascade fashion as part of an immune response, serving a complementary role alongside the antibody immune response, having an important role in host defense against microorganisms and in the modulation of inflammatory reactions. Complement is activated by three pathways: the classical pathway, alternative pathway and lectin pathway, in which each pathway initially involves different proteins, but all pathways converge with the cleavage of complement component C3. C3 is cleaved into C3a, which promotes inflammation and recruits circulating immune cells, while C3b forms a complex with other components to initiate a cascade of reactions among the later components of the complement system. C3b complexes with other complement components to form the C5-convertase complex. Complement component C5 is cleaved by the C5-convertase complex into C5a and C5b. C5a promotes inflammation, such as by acting as a chemoattractant for inflammatory cells. C5b remains attached to the cell surface where it triggers the formation of the membrane attack complex (MAC). The MAC is a hydrophilic pore that spans the membrane and promotes the free flow of fluid into and out of the cell, thereby destroying it.

Complement system dysregulation can result in different pathological conditions. Cells express proteins that protect them from the effects of the complement cascade to ensure that targets of the complement system are limited to pathogenic cells. Many complement-related disorders and diseases are associated with abnormal destruction of self cells by the complement cascade. An example of such disorder is paroxysmal nocturnal hemoglobinuria (PNH), PNH can arise from a genetic mutation that depletes one or more cytoprotective proteins that prevent destruction of red blood cells platelets and other blood cells from complement-mediated attack and can be characterized by hemolytic anemia (a decreased number of RBCs due to cell lysis), hemoglobinuria (hemoglobin in the urine due to RBC lysis), and/or hemoglobinemia (free hemoglobin in the bloodstream due to RBC lysis).

A therapeutic agent that can be used to treat a complement-related disorder is an agent that can inhibit C5 cleavage, such as an antibody that binds complement C5. One example of such a therapeutic agent is eculizumab, which is marketed as Soliris® (Alexion Pharmaceuticals, Inc., New Haven, CT). Another example is ravulizumab, which is marketed as Ultomiris® (Alexion Pharmaceuticals, Inc., New Haven, CT).

Molecular attributes define the physicochemical properties of therapeutic biological molecules, and can therefore impact the drug safety and efficacy. The levels of attributes critical to the drug quality, or critical quality attributes (CQAs), are explicitly defined by the product purity specifications subject to extensive regulatory reviews. There is a need for identifying and producing anti-C5 antibody compositions having suitable CQA profiles so that clinically acceptable compositions may be made available to patients.

### SUMMARY

Pharmaceutical compositions and methods are described herein. By way of example, pharmaceutical compositions, methods comprising administering the pharmaceutical composition, and methods of manufacturing the pharmaceutical composition are described in the following paragraphs.

1. In some embodiments, a pharmaceutical composition is described. The pharmaceutical composition comprises an anti-C5 antibody comprising a heavy chain comprising a Complementarity Determining Region (CDR)H1, CDRH2, and CDRH3, in which the amino acid sequence of the CDRH1, CDRH2 and CDRH3 is SEQ ID NO: 1, 2 and 3, respectively, or SEQS ID NO: 4, 5, and 3, respectively; and a light chain comprising a CDRL1, CDRL2, and CDRL3, in which the amino acid sequence of the CDRL1, CDRL2, and CDRL3 is SEQ ID NO: 12, 13 and 14, respectively. The heavy chain may comprise: (a) Methionine 253 (M253) (EU numbering), wherein greater than 6% and no more than 20% of the anti-C5 antibody heavy chains of the composition comprise oxidized M253; and/or (b) M359 (EU numbering), wherein greater than 2% and no more than 14% of the anti-C5 antibody heavy chains of the composition comprise oxidized M359; and/or (c) M429 (EU numbering), wherein greater than 2% and no more than 14% of the anti-C5 antibody heavy chains of the composition comprise oxidized M429; and/or (d) W33 (EU numbering), wherein greater than 1% and no more than 13% of the anti-C5 antibody heavy chains of the composition comprise oxidized W33; and/or (e) W107 (EU numbering), wherein greater than 4% and no more than 14% of the anti-C5 antibody heavy chains of the composition comprise oxidized W107.

2. In some embodiments, for any pharmaceutical composition described herein, including the pharmaceutical composition of paragraph 1, the heavy chain comprises: (a) M253 (EU numbering), wherein greater than 7% and no more than 20% of the anti-C5 antibody heavy chains of the composition comprise oxidized M253; and/or (b) M359 (EU numbering), wherein greater than 5% and no more than 14% of the anti-C5 antibody heavy chains of the composition comprise oxidized M359; and/or (c) M429 (EU numbering), wherein greater than 5% and no more than 14% of the anti-C5 antibody heavy chains of the composition comprise oxidized M429; and/or (d) W33 (EU numbering), wherein greater than 2% and no more than 13% of the anti-C5 antibody heavy chains of the composition comprise oxidized W33; and/or (e) W107 (EU numbering), wherein greater than 5% and no more than 14% of the anti-C5 antibody heavy chains of the composition comprise oxidized W107.

3. In some embodiments, for any pharmaceutical composition described herein, including the pharmaceutical composition of paragraph 1, the heavy chain comprises: (a) M253 (EU numbering), wherein greater than 8% and no more than 20% of the anti-C5 antibody heavy chains of the composition comprise oxidized M253; and/or (b) M359 (EU numbering), wherein greater than 6% and no more than 14% of the anti-C5 antibody heavy chains of the composition comprise oxidized M359; and/or (c) M429 (EU numbering), wherein greater than 6% and no more than 14% of the anti-C5 antibody heavy chains of the composition comprise oxidized M429; and/or (d) W33 (EU numbering), wherein greater than 3% and no more than 13% of the anti-C5 antibody heavy chains of the composition comprise oxidized W33; and/or (e) W107 (EU numbering), wherein greater than 6% and no more than 14% of the anti-C5 antibody heavy chains of the composition comprise oxidized W107.

4. In some embodiments, for any pharmaceutical composition described herein, including the pharmaceutical composition of paragraph 1, the heavy chain comprises: (a) M253 (EU numbering), in which greater than 6% and no more than 18% of the anti-C5 antibody heavy chains of the composition comprise oxidized M253; and/or (b) M359 (EU numbering), in which greater than 2% and no more than 4% of the anti-C5 antibody heavy chains of the composition comprise oxidized M359; and/or (c) M429 (EU numbering), in which greater than 2% and no more than 6% of the anti-C5 antibody heavy chains of the composition comprise oxidized M429; and/or (d) W33 (EU numbering), in which greater than 1% and no more than 2% of the anti-C5 antibody heavy chains of the composition comprise oxidized W33; and/or (e) W107 (EU numbering), in which greater than 4% and no more than 11% of the anti-C5 antibody heavy chains of the composition comprise oxidized W107.

5. In some embodiments, for any pharmaceutical composition described herein, including the pharmaceutical composition of paragraph 1, the heavy chain comprises: (a) M253 (EU numbering), in which greater than 6% and no more than 17% of the anti-C5 antibody heavy chains of the composition comprise oxidized M253; and/or (b) M359 (EU numbering), in which greater than 2% and no more than 4% of the anti-C5 antibody heavy chains of the composition comprise oxidized M359; and/or (c) M429 (EU numbering), in which greater than 2% and no more than 6% of the anti-C5 antibody heavy chains of the composition comprise oxidized M429; and/or (d) W33 (EU numbering), in which greater than 1% and no more than 2% of the anti-C5 antibody heavy chains of the composition comprise oxidized W33; and/or (e) W107 (EU numbering), in which greater than 4% and no more than 10% of the anti-C5 antibody heavy chains of the composition comprise oxidized W107.

6. In some embodiments, for any pharmaceutical composition described herein, including the pharmaceutical composition of paragraph 1, the heavy chain comprises: (a) M253 (EU numbering), in which greater than 6% and no more than 12% of the anti-C5 antibody heavy chains of the composition comprise oxidized M253; and/or (b) M359 (EU numbering), in which greater than 2% and no more than 3% of the anti-C5 antibody heavy chains of the composition comprise oxidized M359; and/or (c) M429 (EU numbering), in which greater than 2% and no more than 5% of the anti-C5 antibody heavy chains of the composition comprise oxidized M429; and/or (d) W33 (EU numbering), in which greater than 1% and no more than 2% of the anti-C5 antibody heavy chains of the composition comprise oxidized W33; and/or (e) W107 (EU numbering), in which greater than 4% and no more than 8% of the anti-C5 antibody heavy chains of the composition comprise oxidized W107.

7. In some embodiments, for any pharmaceutical composition described herein, including the pharmaceutical composition of paragraph 1, the heavy chain comprises: (a) M253 (EU numbering), wherein greater than 7% and no more than 18% of the anti-C5 antibody heavy chains of the composition comprise oxidized M253; and/or (b) M359 (EU numbering), wherein greater than 3% and no more than 4% of the anti-C5 antibody heavy chains of the composition comprise oxidized M359; and/or (c) M429 (EU numbering), wherein greater than 3% and no more than 6% of the anti-C5 antibody heavy chains of the composition comprise oxidized M429; and/or (d) W33 (EU numbering), wherein greater than 1% and no more than 2% of the anti-C5 antibody heavy chains of the composition comprise oxidized W33; and/or (e) W107 (EU numbering), wherein greater than 5% and no more than 11% of the anti-C5 antibody heavy chains of the composition comprise oxidized W107.

8. In some embodiments, for any pharmaceutical composition described herein, including the pharmaceutical composition of paragraph 1, the heavy chain comprises: (a) M253 (EU numbering), wherein greater than 8% and no more than 18% of the anti-C5 antibody heavy chains of the composition comprise oxidized M253; and/or (b) M359 (EU numbering), wherein greater than 3% and no more than 4% of the anti-C5 antibody heavy chains of the composition comprise oxidized M359; and/or (c) M429 (EU numbering), wherein greater than 4% and no more than 6% of the anti-C5 antibody heavy chains of the composition comprise oxidized M429; and/or (d) W33 (EU numbering), wherein greater than 1% and no more than 2% of the anti-C5 antibody heavy chains of the composition comprise oxidized W33; and/or (e) W107 (EU numbering), wherein greater than 6% and no more than 11% of the anti-C5 antibody heavy chains of the composition comprise oxidized W107.

9. In some embodiments, for any pharmaceutical composition described herein, including the pharmaceutical composition of any one of paragraphs 1-8, the heavy chain comprises: (c) M429 (EU numbering), in which greater than 2% and no more than 6% of the anti-C5 antibody heavy chains of the composition comprise oxidized M429; and/or (e) W107 (EU numbering), in which greater than 6% and no more than 11% of the anti-C5 antibody heavy chains of the composition comprise oxidized W107.

10. In some embodiments, for any pharmaceutical composition described herein, including the pharmaceutical composition of any one of paragraphs 1-9, the heavy chain comprises: (a) and (b); or (a) and (c); or (a) and (d); or (a) and (e); or (b) and (c); or (b) and (d); or (d) and (e); or (c) and (d); or (c) and (e); or (d) and (e).

11. In some embodiments, for any pharmaceutical composition described herein, including the pharmaceutical composition of any one of paragraphs 1-10, the heavy chain comprises (a), (b), and (c); or (a), (b), (c), and (d).

12. In some embodiments, for any pharmaceutical composition described herein, including the pharmaceutical composition of any one of paragraphs 1-11, the anti-C5 antibody is authorized for administration to a human subject by a government regulatory agency.

13. In some embodiments, for any pharmaceutical composition described herein, including the pharmaceutical composition of any one of paragraphs 1-12, the percent oxidized amino acid residue is a ratio of HPLC peak areas of reduced peptide mapping of the anti-C5 antibody.

14. In some embodiments, for any pharmaceutical composition described herein, including the pharmaceutical composition of any one of paragraphs 1-13, the percent oxidized amino acid residue is as determined by reduced peptide mapping comprising: denaturing the anti-C5 antibody at a concentration of 1 mg/ml in 7.5M guanidine HCl, 0.25M Tris HCl, 2 mM EDTA pH 7.5; reducing the denatured anti-C5 antibody in 9 mM Dithiothreitol (DTT) at 27° C.; alkylating the denatured anti-C5 antibody in 16 mM Iodoacetic Acid (IAA) at 27° C.; desalting the alkylated, denatured anti-C5 antibody; digesting 1 mg/ml of the desalted anti-C5 antibody at pH 7.5 with trypsin at 0.08 mg/ml at 37° C., thereby producing peptides; quenching the digest in 0.5% Trifluoroacetic Acid (TFA); separating the peptides by reverse phase high performance liquid chromatography (RP-HPLC) on column comprising C4 chemistry, the separating comprising a mobile phase A of 0.1% TFA in water and mobile phase B of 0.1% TFA in acetonitrile (ACN) on a gradient comprising from 0.5% to 45% mobile phase B from 3 to 93 minutes at a column temperature of 50° C., a sample tray temperature of 8° C., an injection volume of 50 μL, and a flow rate of 200 μL/minute; and detecting the separated peptides by on-line mass spectrometry (MS) and MS/MS. The (%) methionine oxidation is a frequency of the sum of all types of oxidation detected on the same methionine (M) residue; and in which (%) tryptophan oxidation is a frequency of the sum of all types of oxidation detected on the same tryptophan (W) residue.

15. In some embodiments, for any pharmaceutical composition described herein, including the pharmaceutical composition of any one of paragraphs 1-14, the pharmaceutical composition comprises no more than 5.0% high molecular weight (HMW) species of the anti-C5 antibody as determined by Size Exclusion Ultra High-Performance Liquid Chromatography (SE-UHPLC).

16. In some embodiments, for any pharmaceutical composition described herein, including the pharmaceutical composition of any one of paragraphs 1-154, the pharmaceutical composition comprises greater than 1.5%, and no more than 7.0% HMW species of the anti-C5 antibody as determined by SE-UHPLC.

17. In some embodiments, for any pharmaceutical composition described herein, including the pharmaceutical composition of any one of paragraphs 1-15, the pharmaceutical composition comprises at least 1.8%, and no more than 7.0% HMW species as determined by SE-UHPLC.

18. In some embodiments, for any pharmaceutical composition described herein, including the pharmaceutical composition of any one of paragraphs 1-15, the pharmaceutical composition comprises at least 2%, and no more than 7.0% HMW species as determined by SE-UHPLC.

19. In some embodiments, for any pharmaceutical composition described herein, including the pharmaceutical composition of any one of paragraphs 1-15, the pharmaceutical composition comprises at least 1.5%, and no more than 5.0% HMW species as determined by SE-UHPLC.

20. In some embodiments, a pharmaceutical composition is described, comprising: an anti-C5 antibody comprising: a heavy chain comprising a CDRH1, CDRH2, and CDRH3, in which the amino acid sequence of the CDRH1, CDRH2 and CDRH3 is SEQ ID NO: 1, 2 and 3, respectively, or SEQS ID NO: 4, 5, and 3, respectively; and a light chain comprising a CDRL1, CDRL2, and CDRL3, in which the amino acid sequence of the CDRL1, CDRL2, and CDRL3 is SEQ ID NO: 12, 13 and 14, respectively. The pharmaceutical composition comprises no more than 7.0% HMW species anti-C5 antibody as determined by SE-UHPLC. By way of example, the pharmaceutical composition may comprise no more than 5.0% HMW species anti-C5 antibody as determined by SE-UHPLC.

21. In some embodiments, for any pharmaceutical composition described herein, including the pharmaceutical composition of paragraph 20, the pharmaceutical composition comprises greater than 1.5%, and no more than 5.0% HMW species as determined by SE-UHPLC.

22. In some embodiments, for any pharmaceutical composition described herein, including the pharmaceutical composition of any one of paragraphs 1-21, in which the anti-C5 antibody has a relative potency of at least 80%, 81%, 82%, 83%, 84%, 85%, 86%, 87%, 88%, 89%, 90%, 91%, 92%, 93%, 94%, or 95% compared to a reference anti-C5 antibody.

23. In some embodiments, for any pharmaceutical composition described herein, including the pharmaceutical composition of paragraph 22, the relative potency comprises or consists of relative inhibition of terminal complement complex (TCC) formation.

24. In some embodiments, for any pharmaceutical composition described herein, including the pharmaceutical composition of paragraph 22 or 23, the relative potency is as measured by enzyme-linked immunosorbent assay (ELISA) comprising: immobilizing pre-activated zymosan on a substrate, and subsequently blocking unbound surfaces of the substrate with bovine serum albumin; incubating the anti-C5 antibody with normal human serum comprising C5, whereby TCC forms and binds to the zymosan on the substrate; and detecting the TCC with alkaline phosphatase conjugated anti-C5b-9 antibody and an alkaline phosphatase substrate.

25. In some embodiments, for any pharmaceutical composition described herein, including the pharmaceutical composition of any one of paragraphs 1-24, the anti-C5 antibody has a half-life of at least 190 hours, such as 190-352 hours, such as about 272 hours.

26. In some embodiments, for any pharmaceutical composition described herein, including the pharmaceutical composition of paragraph 25, the half-life is as determined by quantification of total (bound+free) anti-C5 antibody in serum by enzyme linked immunosorbent assay (ELISA) comprising human C5 immobilized on a substrate, for a dose of anti-C5 antibody of 300 mg-1200 mg intravenously.

27. In some embodiments, for any pharmaceutical composition described herein, including the pharmaceutical composition of paragraph 25, the half-life is as determined by quantification of free anti-C5 antibody in serum by an electrochemiluminescence (ECL) assay comprising two anti-idiotypic antibodies (one is immobilized on a substrate and one is used for detection), for a dose of anti-C5 antibody of 300 mg-1200 mg intravenously.

28. In some embodiments, for any pharmaceutical composition described herein, including the pharmaceutical composition of any one of paragraphs 25-27, an increase of high mannose species from 0% to 10% results in no more than 3.4% increase or no more than a 4.5% increase in clearance of anti-C5 antibody species, such as no more than 3.4% (for PNH patients) or no more than 4.5% (for aHUS patients).

29. In some embodiments, for any pharmaceutical composition described herein, including the pharmaceutical composition of any one of paragraphs 1-28, the anti-C5 antibody comprises a heavy chain variable region having the amino acid sequence of SEQ ID NO: 6 or 7.

30. In some embodiments, for any pharmaceutical composition described herein, including the pharmaceutical composition of any one of paragraphs 1-29, the anti-C5 antibody comprises a heavy chain constant region having the amino acid sequence of SEQ ID NO: 8 or 9.

31. In some embodiments, for any pharmaceutical composition described herein, including the pharmaceutical composition of any one of paragraphs 1-30, the anti-C5 antibody comprises a heavy chain having the amino acid sequence of SEQ ID NO: 10 or 11.

32. In some embodiments, for any pharmaceutical composition described herein, including the pharmaceutical composition of any one of paragraphs 1-31, the anti-C5 antibody comprises a light chain variable region having the amino acid sequence of SEQ ID NO: 15.

33. In some embodiments, for any pharmaceutical composition described herein, including the pharmaceutical composition of any one of paragraphs 1-32, the anti-C5 antibody comprises a light chain constant region having the amino acid sequence of SEQ ID NO: 16.

34. In some embodiments, for any pharmaceutical composition described herein, including the pharmaceutical composition of any one of paragraphs 1-32, the anti-C5 antibody comprises a light chain having the amino acid sequence of SEQ ID NO: 17.

35. In some embodiments, for any pharmaceutical composition described herein, including the pharmaceutical composition of any one of paragraphs 1-34, the pharmaceutical composition further comprises a buffer.

36. In some embodiments, for any pharmaceutical composition described herein, including the pharmaceutical composition of paragraph 35, in which the buffer comprises acetate, optionally between 5 mM and 20 mM.

37. In some embodiments, for any pharmaceutical composition described herein, including the pharmaceutical composition of any one of paragraphs 35 or 36, the pharmaceutical composition further comprises a stabilizer.

38. In some embodiments, for any pharmaceutical composition described herein, including the pharmaceutical composition of paragraph 37, the stabilizer comprises sorbitol, optionally about 5% (w/v).

39. In some embodiments, for any pharmaceutical composition described herein, including the pharmaceutical composition of any one of paragraphs 1-38, the pharmaceutical composition further comprises a chelating agent.

40 In some embodiments, for any pharmaceutical composition described herein, including the pharmaceutical composition of paragraph 39, the chelating agent comprises ethylenediaminetetraacetic acid (EDTA), optionally at a concentration of about 0.01 mM to about 0.05 mM, such as 0.038 mM to 0.063 mM.

41. In some embodiments, for any pharmaceutical composition described herein, including the pharmaceutical composition of any one of paragraphs 1-40, the pharmaceutical composition comprises 300-900 mg of the anti-C5 antibody. The composition may comprise or consist of a unit dose of the anti-C5 antibody.

42. In some embodiments, for any pharmaceutical composition described herein, including the pharmaceutical composition of any one of paragraphs 1-40, the pharmaceutical composition comprises 300 mg of the anti-C5 antibody. The composition may comprise or consist of a unit dose of the anti-C5 antibody.

43. In some embodiments, for any pharmaceutical composition described herein, including the pharmaceutical composition of any one of paragraphs 1-40, the pharmaceutical composition comprises 900 mg of the anti-C5 antibody. The composition may comprise or consist of a unit dose of the anti-C5 antibody.

44. In some embodiments, for any pharmaceutical composition described herein, including the pharmaceutical composition of any one of paragraphs 1-43, the pharmaceutical composition is for medical use.

45. In accordance with some embodiments, described herein is a method comprising administering the pharmaceutical composition of any of paragraphs 1-44 to a subject in need of treatment by the anti-C5 antibody.

46. In some embodiments, for any method comprising administering a pharmaceutical composition described herein, including the method of paragraph 45, the subject has myasthenia gravis, paroxysmal nocturnal hemoglobinuria, neuromyelitis optica spectrum disorder, or atypical hemolytic uremic syndrome.

47. In some embodiments, for any method comprising administering a pharmaceutical composition described herein, including the method of paragraph 45 or 46, the anti-C5 antibody is administered to the subject at a dose of 600 mg-1200 mg or 300 mg-1200 mg, such as 300 mg, 600 mg, 900 mg, or 1200 mg. The pharmaceutical composition may be administered intravenously.

48. In some embodiments, for any method comprising administering a pharmaceutical composition described herein, including the method of paragraph 45 or 46, the anti-C5 antibody is administered at a dose of:
(a) 600 mg weekly for 4 weeks, followed by a 900 mg dose 1 week later, followed by 900 mg every 2 weeks thereafter; or
(b) 900 mg weekly for 4 weeks, followed by a 1200 mg dose 1 week later, followed by 1200 mg every 2 weeks thereafter.

49. In some embodiments, for any method comprising administering a pharmaceutical composition described herein, including the method of paragraph 45 or 46, the anti-C5 antibody is administered at a dose of 300 mg. 50. In some embodiments, a method of manufacturing the pharmaceutical composition of any of paragraphs 1-49 is described. The method comprises providing a composition comprising the anti-C5 antibody; determining percentage of heavy chains of the anti-C5 antibody of the composition that comprise: (a) M253 (EU numbering) oxidation, (b) M359 (EU numbering) oxidation, (c) M429 (EU numbering) oxidation, (d) W33 (EU numbering) oxidation; and/or (e) W107 (EU numbering) oxidation. The method further comprises manufacturing the composition for pharmaceutical use if the heavy chain comprises: (a) M253 (EU numbering), wherein greater than 6% and no more than 20% of the anti-C5 antibody heavy chains of the composition comprise oxidized M253; and/or (b) M359 (EU numbering), wherein greater than 2% and no more than 14% of the anti-C5 antibody heavy chains of the composition comprise oxidized M359; and/or (c) M429 (EU numbering), wherein greater than 2% and no more than 14% of the anti-C5 antibody heavy chains of the composition comprise oxidized M429; and/or (d) W33 (EU numbering), wherein greater than 1% and no more than 13% of the anti-C5 antibody heavy chains of the composition comprise oxidized W33; and/or (e) W107 (EU numbering), wherein greater than 4% and no more than 14% of the anti-C5 antibody heavy chains of the composition comprise oxidized W107.

51. In some embodiments, for any method of manufacturing described herein, including the method of paragraph 50, the composition is manufactured for pharmaceutical use if the heavy chain comprises: (a) M253 (EU numbering), in which greater than 6% and no more than 18% of the anti-C5 antibody heavy chains of the composition comprise oxidized M253; and/or (b) M359 (EU numbering), in which greater than 2% and no more than 4% of the anti-C5 antibody heavy chains of the composition comprise oxidized M359; and/or (c) M429 (EU numbering), in which greater than 2% and no more than 6% of the anti-C5 antibody heavy chains of the composition comprise oxidized M429; and/or (d) W33 (EU numbering), in which greater than 1% and no more than 2% of the anti-C5 antibody heavy chains of the composition comprise oxidized W33; and/or (e) W107 (EU numbering), in which greater than 4% and no more than 11% of the anti-C5 antibody heavy chains of the composition comprise oxidized W107. For example, the composition for pharmaceutical use if the heavy chain comprises 52. In some embodiments, for any method of manufacturing described herein, including the method of paragraph 50, the composition is manufactured for pharmaceutical use if the heavy chain comprises: (a) M253 (EU numbering), in which greater than 6% and no more than 17% of the anti-C5 antibody heavy chains of the composition comprise oxidized M253; and/or (b) M359 (EU numbering), in which greater than 2% and no more than 4% of the anti-C5 antibody heavy chains of the composition comprise oxidized M359; and/or (c) M429 (EU numbering), in which greater than 2% and no more than 6% of the anti-C5 antibody heavy chains of the composition comprise oxidized M429; and/or (d) W33 (EU numbering), in which greater than 1% and no more than 2% of the anti-C5 antibody heavy chains of the composition comprise oxidized W33; and/or (e) W107 (EU numbering), in which greater than 4% and no more than 10% of the anti-C5 antibody heavy chains of the composition comprise oxidized W107.

53. In some embodiments, for any method of manufacturing described herein, including the method of paragraph 50, the composition is manufactured for pharmaceutical use if the heavy chain comprises: (a) M253 (EU numbering), in which greater than 6% and no more than 12% of the anti-C5 antibody heavy chains of the composition comprise oxidized M253; and/or (b) M359 (EU numbering), in which greater than 2% and no more than 3% of the anti-C5 antibody heavy chains of the composition comprise oxidized M359; and/or (c) M429 (EU numbering), in which greater than 2% and no more than 5% of the anti-C5 antibody heavy chains of the composition comprise oxidized M429; and/or (d) W33 (EU numbering), in which greater than 1% and no more than 2% of the anti-C5 antibody heavy chains of the composition comprise oxidized W33; and/or (e) W107 (EU numbering), in which greater than 4% and no more than 8% of the anti-C5 antibody heavy chains of the composition comprise oxidized W107.

54. In some embodiments, for any method of manufacturing described herein, including the method of paragraph 50, the composition is manufactured for pharmaceutical use if the heavy chain comprises: (a) M253 (EU numbering), wherein greater than 7% and no more than 18% of the anti-C5 antibody heavy chains of the composition comprise oxidized M253; and/or (b) M359 (EU numbering), wherein greater than 3% and no more than 4% of the anti-C5 antibody heavy chains of the composition comprise oxidized M359; and/or (c) M429 (EU numbering), wherein greater than 3% and no more than 6% of the anti-C5 antibody heavy chains of the composition comprise oxidized M429; and/or (d) W33 (EU numbering), wherein greater than 1% and no more than 2% of the anti-C5 antibody heavy chains of the composition comprise oxidized W33; and/or (e) W107 (EU numbering), wherein greater than 5% and no more than 11% of the anti-C5 antibody heavy chains of the composition comprise oxidized W107.

55. In some embodiments, for any method of manufacturing described herein, including the method of paragraph 50, the composition is manufactured for pharmaceutical use if the heavy chain comprises: (a) M253 (EU numbering), wherein greater than 8% and no more than 18% of the anti-C5 antibody heavy chains of the composition comprise oxidized M253; and/or (b) M359 (EU numbering), wherein greater than 3% and no more than 4% of the anti-C5 antibody heavy chains of the composition comprise oxidized M359; and/or (c) M429 (EU numbering), wherein greater than 4% and no more than 6% of the anti-C5 antibody heavy chains of the composition comprise oxidized M429; and/or (d) W33 (EU numbering), wherein greater than 1% and no more than 2% of the anti-C5 antibody heavy chains of the composition comprise oxidized W33; and/or (e) W107 (EU numbering), wherein greater than 6% and no more than 11% of the anti-C5 antibody heavy chains of the composition comprise oxidized W107.

56. In some embodiments, for any method of manufacturing described herein, including the method of any one of paragraphs 50-55, the composition is manufactured for pharmaceutical use if the heavy chain comprises: (c) M429 (EU numbering), in which greater than 2% and no more than 6% of the anti-C5 antibody heavy chains of the composition comprise oxidized M429; and/or (e) W107 (EU numbering), in which greater than 6% and no more than 11% of the anti-C5 antibody heavy chains of the composition comprise oxidized W107.

57. In some embodiments, for any method of manufacturing described herein, including the method of any one of paragraphs 50-56, the composition is manufactured for pharmaceutical use if the heavy chain comprises: (a) and (b); or (a) and (c); or (a) and (d); or (a) and (e); or (b) and (c); or (b) and (d); or (d) and (e); or (c) and (d); or (c) and (e); or (d) and (e).

58. In some embodiments, for any method of manufacturing described herein, including the method of any one of paragraphs 50-57, the composition is manufactured for pharmaceutical use if the heavy chain comprises (a), (b), and (c); or (a), (b), (c), and (d).

58. In some embodiments, for any method of manufacturing described herein, including the method of any one of paragraphs 50-58, the composition is manufactured for pharmaceutical use if the composition comprises no more than 5.0% HMW species of anti-C5 antibody as determined by SE-UHPLC.

59. In some embodiments, for any method of manufacturing described herein, including the method of any one of paragraphs 50-58, the composition is manufactured for pharmaceutical use if the composition comprises greater than 1.5% HMW species of anti-C5 antibody, and no more than 5.0% HMW species of anti-C5 antibody as determined by SE-UHPLC.

60. In some embodiments, for any method of manufacturing described herein, including the method of any one of paragraphs 50-59, the composition is manufactured in a 300 mg unit dose of anti-C5 antibody.

61. In, some embodiments, for any method of manufacturing described herein, including the method of any one of paragraphs 50-59, the composition is manufactured in a 900 mg unit dose of anti-C5 antibody.

62. In some embodiments, for any pharmaceutical composition described herein, including the pharmaceutical composition of any one of paragraphs 1-44, the anti-C5 antibody is eculizumab.

63. In some embodiments, for any method described herein, including the method of any one of paragraphs 45-61, the anti-C5 antibody is eculizumab.

### DETAILED DESCRIPTION

Described herein are compositions comprising anti-C5 antibody such as eculizumab, and having profiles of molecular attributes suitable for medical use. Several molecular attributes of eculizumab, including oxidation of heavy chain Met253, Met359, Met429, Trp33, and Trp107 (EU numbering) have been identified as critical quality attributes. Oxidation of Trp33 and Trp107 has been shown to impact potency. Without being limited by theory, it is noted that Trp33 and Trp107 are located in heavy chain CDR regions, and are contemplated to be directly involved in C5 binding of the anti-C5 antibody (See, Schatz-Jakobsen et al. (2016), J. Immunol 197:337-44 “Structural Basis for Eculizumab-Mediated Inhibition of the Complement Terminal Pathway”, reporting that HCDR3 residues 101-107 form a binding pocket for C5 residues Y917 and F918). Reported herein is analysis of molecular attribute profiles of eculizumab compositions determined to have an acceptable clinical profile, including potency profile. Molecular attributes of eculizumab compositions were measured throughout product shelf life, including up to end-of-shelf. Unexpectedly, it was determined that clinical formulations of eculizumab could comprise molecular attribute ranges considerably broader than those that had conventionally been specified for eculizumab, thus enhancing the options for efficiently manufacturing safe and effective anti-C5 antibody, while minimizing waste. In some embodiments, the anti-C5 antibody comprises or consists of eculizumab.

### Anti-C5 Antibody

Anti-C5 antibodies are described herein, and may be used in any of the compositions or methods described herein. The anti-C5 antibody may bind to C5, and inhibit C5 cleavage. Unless expressly stated otherwise, all amino acid numbering for antibodies described herein will refer to EU numbering.

The anti-C5 antibody may comprise heavy chain CDRs having the amino acid sequence of GYIFSNYWIQ (SEQ ID NO: 1) for CDRH1, the amino acid sequence of EILPGSGSTEYTENFKD (SEQ ID NO: 2) for CDRH2, and the amino acid sequence of YFFGSSPNWYFDV (SEQ ID NO: 3) for CDRH3. The antibody may further comprise light chain CDRs having the amino acid sequence of GASENIYGALN (SEQ ID NO: 12) for CDRL1, the amino acid sequence of GATNLAD (SEQ ID NO: 13) for CDRL2, and the amino acid sequence of QNVLNTPLT (SEQ ID NO: 14) for CDRL3. In some embodiments, the anti-C5 antibody comprises heavy chain CDRs having the amino acid sequence of the amino acid sequence of GHIFSNYWIQ (SEQ ID NO: 4) for CDRH1, the amino acid sequence of EILPGSGHTEYTENFKD (SEQ ID NO: 5) for CDRH2, and the amino acid sequence of SEQ ID NO: 3 for CDRH3.

In some embodiments, the anti-C5 antibody comprises a heavy chain variable domain having an amino acid sequence of:

(SEQ ID NO: 6)
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSS

In some embodiments, the anti-C5 antibody comprises a heavy chain variable region having an amino acid sequence of:

(SEQ ID NO: 7)
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSS

In some embodiments, the anti-C5 antibody comprises a heavy chain constant region having an amino acid sequence of:

(SEQ ID NO: 8)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVMHEALHNHYTQKSLSLSLGK

In some embodiments, the anti-C5 antibody comprises a heavy chain constant region having an amino acid sequence of:

(SEQ ID NO: 9)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVLHEALHSHYTQKSLSLSLGK

In some embodiments, the anti-C5 antibody comprises a heavy chain having an amino acid sequence of:

(SEQ ID NO: 10)
QVQLVQSGAEVKKPGASVKVSCKASGYIFSNYWIQWVRQAPGQGLEWMGE
ILPGSGSTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK

In some embodiments, the anti-C5 antibody comprises a heavy chain having an amino acid sequence of:

(SEQ ID NO: 11)
QVQLVQSGAEVKKPGASVKVSCKASGHIFSNYWIQWVRQAPGQGLEWMGE
ILPGSGHTEYTENFKDRVTMTRDTSTSTVYMELSSLRSEDTAVYYCARYF
FGSSPNWYFDVWGQGTLVTVSSASTKGPSVFPLAPCSRSTSESTAALGCL
VKDYFPEPVTVSWNSGALTSGVHTFPAVLQSSGLYSLSSVVTVPSSNFGT
QTYTCNVDHKPSNTKVDKTVERKCCVECPPCPAPPVAGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVLHEALHSHYTQKSLSLSLGK

In some embodiments, the anti-C5 antibody comprises light chain CDRs having the amino acid sequence of GASENIYGALN (SEQ ID NO: 12) for CDRL1, the amino acid sequence of GATNLAD (SEQ ID NO: 13) for CDRL2, and the amino acid sequence of QNVLNTPLT (SEQ ID NO: 14) for CDRL3.

In some embodiments, the anti-C5 antibody comprises a light chain variable region having an amino acid sequence of:

(SEQ ID NO: 15)
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIK

In some embodiments, the anti-C5 antibody comprises a light chain constant region having an amino acid sequence of:

(SEQ ID NO: 16)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

In some embodiments, the anti-C5 antibody comprises a light chain having an amino acid sequence of:

(SEQ ID NO: 17)
DIQMTQSPSSLSASVGDRVTITCGASENIYGALNWYQQKPGKAPKLLIYG
ATNLADGVPSRFSGSGSGTDFTLTISSLQPEDFATYYCQNVLNTPLTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

In some embodiments, the anti-C5 antibody comprises a heavy chain comprising CDRH1-3, wherein CDRH1-3 comprises the amino acid sequences of SEQ ID NOs: 1-3, respectively; and a light chain comprising CDRL1-3, wherein CDRL1-3 comprises the amino acid sequences of SEQ ID NOs: 12-14, respectively. In some embodiments, the anti-C5 antibody comprises a heavy chain comprising CDRH1-3, wherein CDRH1-3 comprises the amino acid sequences of SEQ ID NOs: 4, 5, and 3, respectively; and a light chain comprising CDRL1-3, wherein CDRL1-3 comprises the amino acid sequences of SEQ ID NOs: 12-14, respectively.

In some embodiments, the anti-C5 antibody comprises a heavy chain comprising a variable region comprising the amino acid sequence of SEQ ID NO: 6; and a light chain comprising a variable region comprising the amino acid sequence of SEQ ID NO: 15. In some embodiments, the anti-C5 antibody comprises a heavy chain comprising a variable region comprising the amino acid sequence of SEQ ID NO: 7; and a light chain comprising a variable region comprising the amino acid sequence of SEQ ID NO: 15.

In some embodiments, the anti-C5 antibody comprises a heavy chain comprising a variable region and a constant region comprising the amino acid sequences of SEQ ID NOs: 6 and 8, respectively; and a light chain comprising a variable region and a constant region comprising the amino acid sequences of SEQ ID NOs: 15 and 16, respectively. In some embodiments, the anti-C5 antibody comprises a heavy chain comprising a variable region and a constant region comprising the amino acid sequences of SEQ ID NOs: 7 and 9, respectively; and a light chain comprising a variable region and a constant region comprising the amino acid sequences of SEQ ID NOs: 15 and 16, respectively.

In some embodiments, the anti-C5 antibody comprises a heavy chain comprising the amino acid sequence of SEQ ID NO: 10 and a light chain comprising the amino acid sequence of SEQ ID NO: 17. In some embodiments, the anti-C5 antibody comprises a heavy chain comprising the amino acid sequence of SEQ ID NO: 11 and a light chain comprising the amino acid sequence of SEQ ID NO: 17. In some embodiments, the anti-C5 antibody is eculizumab. In some embodiments, the anti-C5 antibody comprises a heavy chain comprising constant regions sequences of an IgG2, IgG4, or hybrid IgG2/IgG4 isotype. In some embodiments, the anti-C5 antibody comprises a heavy chain comprising constant regions sequences of a hybrid IgG2/IgG4 isotype, such as CH1 and Hinge of IgG2 and CH2 and CH3 of IgG4.

### Molecular Attributes

Described herein are molecular attributes of anti-C5 antibodies. “Molecular attributes” and variations of this root term has its ordinary and customary meaning as would be understood by one of ordinary skill in the art in view of this disclosure. It refers to a chemically or physically changed structure on a macromolecule, such as an antibody, and may be characterized in terms of its physicochemical identity or attribute type and location within the sequence of the macromolecule, e.g., the position of the amino acid on which the attribute is present. For example, asparagine and glutamine residues are susceptible to deamidation. An oxidized tryptophan at position 107 on the Heavy Chain (which is in the HCDR3 region) of a therapeutic protein amino acid sequence is an example of a molecular attribute. Exemplary molecular attribute types are described herein. For conciseness, molecular attributes may be referred to herein simply as “attributes.” The levels of attributes critical to the drug quality, or critical quality attributes (CQAs), may be explicitly defined by the product purity specifications. These specifications are typically subject to extensive regulatory reviews. In some embodiments, a specification may set the permissible levels of one or more molecular attributes in the manufacture of a biological therapy.

Examples of molecular attributes include methionine oxidation (Met+Ox) and tryptophan oxidation (Trp+Ox). In some compositions and methods described herein, the anti-C5 antibodies comprise methionine oxidation (Met+Ox), for example oxidation of M253, M359, and/or M429 of the heavy chain of the anti-C5 antibody, such as eculizumab. In some compositions and methods described herein, the anti-C5 antibodies comprise tryptophan oxidation (Trp+Ox), for example oxidation of W33 and/or W107 of the heavy chain of the anti-C5 antibody, such as eculizumab. In some embodiments, the molecular attributes include oxidation of M253 and M359; or M253 and M429; or M359 and M429; or M253, M359, and M429; or M253 and W33; or M359 and W33; or M429 and W33; M253 and W107; or M359 and W107; or M429 and W107; or M253, W33 and W107; or M359, W33, and W107; or M429, W33 and W107; or M253 and M359 and W33; or M253 and M429 and W33; or M359 and M429 and W33; or M253, M359, M429, and W33; or M253 and M359 and W107; or M253 and M429 and W107; or M359 and M429 and W107; or M253, M359, M429, and W107; or M253 and M359, W33 and W107; or M253, M429, W33 and W107; or M359, M429, W33 and W107; or M253, M359, M429, W33, and W107.

The percent oxidized amino acid residue (such as Met+Ox or Trp+Ox) in a composition comprising anti-C5 antibodies as described herein may be determined by reduced peptide mapping. The reduced peptide mapping may comprise reducing a sample comprising the anti-C5 antibody with dithiothreitol (DTT) in denaturant and alkylating with iodoacetic acid (IAA). Excess reagents may be removed by size exclusion-based desalting columns. Digestion with trypsin may be carried out at 37° C. for 1 h. Peptides from the digest may be separated by reverse phase ultra high performance liquid chromatography (RP-UHPLC) in a trifluoroacetic acid/acetonitrile (TFA/ACN) gradient with on-line MS and MS/MS data collection. The percent oxidized amino acid residue may be a ratio of RP-UHPLC peak areas from reduced peptide mapping of the anti-C5 antibody.

By way of example, for any composition of method described herein, the percent oxidized amino acid residue may be determined by reduced peptide mapping comprising denaturing the anti-C5 antibody at a concentration of 1 mg/ml in 7.5M guanidine HCl, 0.25M Tris HCl, 2 mM EDTA pH 7.5. The reduced peptide mapping may further comprise reducing the denatured anti-C5 antibody in 9 mM Dithiothreitol (DTT) at 27° C. The reduced peptide mapping may further comprise alkylating the denatured anti-C5 antibody in 16 mM Iodoacetic Acid (IAA) at 27° C. The reduced peptide mapping may further comprise desalting the alkylated, denatured anti-C5 antibody. The reduced peptide mapping may further comprise digesting 1 mg/ml of the desalted anti-C5 antibody at pH 7.5 with trypsin at 0.08 mg/ml at 37° C., thus producing peptides. The reduced peptide mapping may further comprise quenching the digest in 0.5% Trifluoroacetic Acid (TFA). The reduced peptide mapping may further comprise separating the peptides by reverse phase ultra high performance liquid chromatography (RP-UPLC) on column comprising C4 chemistry, the separating comprising a mobile phase A of 0.1% TFA in water and mobile phase B of 0.1% TFA in acetonitrile (ACN) on a gradient comprising from 0.5% to 45% mobile phase B from 3 to 93 minutes at column temperature of 50° C., a sample tray temperature of 8° C., an injection volume of 50 μL, and a flow rate of 200 μL/minute. The reduced peptide mapping may further comprise detecting the separated peptides by on-line mass spectrometry (MS) and MS/MS. The (%) methionine oxidation may be calculated as a frequency of the sum of all types of oxidation detected on the same methionine (M) residue. The (%) tryptophan oxidation may be calculated as a frequency of the sum of all types of oxidation detected on the same tryptophan (W) residue. It is contemplated that some implementations of reduced peptide mapping may use a sample tray temperature of 2-8° C.

High molecular weight (HMW) species is a molecular attribute that has been reported to have potential impacts on safety and/or potency of some therapeutic antibodies. HMW species for anti-C5 antibody of pharmaceutical compositions and methods as described herein may be as determined by Size Exclusion Chromatography, such as Size Exclusion Ultra-High Performance Liquid Chromatography (SE-UHPLC). The HMW species refer to peaks with a greater molecular weight than a single molecule of the native anti-C5 antibody, such as dimers of anti-C5 antibody, trimers of anti-C5 antibody, and the like. Conventionally, the specification for HMW species for eculizumab has been set as ≤1.5%. However, it has been determined herein that clinically suitable pharmaceutical compositions of anti-C5 antibody such as eculizumab may comprise HMW species without an impact on safety or potency (See Example 3). Accordingly, it is contemplated that pharmaceutical compositions as described herein may comprise greater than 1.5% HMW species of anti-C5 antibody (as determined by SE-UHPLC) and be suitable for medical use. In some embodiments, the pharmaceutical composition (or the pharmaceutical composition of the method) as described herein comprises greater than 1.5% HMW species of anti-C5 antibody, but no more than 7% HMW species of anti-C5 antibody as determined by SE-UHPLC. In some embodiments, the pharmaceutical composition (or the pharmaceutical composition of the method) as described herein comprises greater than 1.5% HMW species of anti-C5 antibody, but no more than 5% HMW species of anti-C5 antibody as determined by SE-UHPLC. For example, the pharmaceutical composition (or the pharmaceutical composition of the method) may comprise greater than 1.5% HMW species of anti-C5 antibody, but no more than 2%, 1.9%, 1.8%, or 1.7% HMW species of anti-C5 antibody as determined by SE-UHPLC. For example, the pharmaceutical composition (or the pharmaceutical composition of the method) may comprise at least 1.7% HMW species of anti-C5 antibody, but no more than 2%, 1.9%, 1.8%, or 1.7% HMW species of anti-C5 antibody as determined by SE-UHPLC. For example, the pharmaceutical composition (or the pharmaceutical composition of the method) may comprise at least 1.8% HMW species of anti-C5 antibody, but no more than 2% HMW species of anti-C5 antibody as determined by SE-UHPLC.

### Potency

Anti-C5 antibodies as described herein bind to human complement component 5 (C5) with high affinity and inhibit cleavage of C5, thereby blocking pro-inflammatory and cytolytic effects of terminal complement activation. Terminal complement activation generates the terminal complement complex (TCC) which results in the presentation of a new epitope (neo-antigen, C5b-9) that is distinct from each individual complement protein. The amount of TCC generated is proportional to the functional activity of complement. It will be understood that relative potency of an anti-C5 antibody as described herein may be determined from a relative amount of inhibition of TCC formation compared to a reference standard anti-C5 antibody. As such, it will be appreciated that it is possible for an anti-C5 antibody to have a relative potency of greater than 100%. Potency of the anti-C5 antibodies described herein may be assessed by incubating the anti-C5 antibody with normal human serum, containing human C5, and a complement activator, zymosan, and detecting the amount of TCC (which is indicative of the ability of the anti-C5 antibody to prevent the formation of the TCC). For methods and pharmaceutical compositions described herein, the amount of inhibition of TCC formation refers to an amount as measured by an enzyme-linked immunosorbent assay (ELISA) comprising: immobilizing pre-activated zymosan on a substrate and subsequently blocking unbound surfaces of the substrate with bovine serum albumin (BSA); incubating the anti-C5 antibody with normal human serum comprising C5, whereby TCC forms, as a result of zymosan induced complement activation, and binds to the zymosan on the substrate; and detecting the TCC with an alkaline phosphatase conjugated anti-C5b-9 antibody and an alkaline phosphatase substrate. An example ELISA assay is described in Example 1. Suitable anti-C5 antibodies may comprise Met+Ox, Trp+Ox, or combinations of Met+Ox and Trp+Ox as described herein, and have a relative potency of at least about 80%. Such antibodies may be for medical use. By way of example, an anti-C5 antibody may have a relative potency of at least about 80%, 81%, 82%, 83%, 84%, 85%, 86%, 87%, 88%, 89%, 90%, 91%, 92%, 93%, 94%, or 95%, such as 80%-125%, 80%-115%, 80%-100%, 81%-125%, 81%-115%, 81%-100%, 85%-125%, 85%-115%, 85%-100%, 90%-125%, 90%-115%, or 90%-100%. The anti-C5 antibody may be suitable for medical use.

### Pharmaceutical Compositions

Described herein are pharmaceutical compositions comprising anti-C5 antibody. The anti-C5 antibody of the pharmaceutical composition may comprise a heavy chain comprising a CDRH1, CDRH2, and CDRH3, in which the amino acid sequence of the CDRH1, CDRH2 and CDRH3 is SEQ ID NO: 1, 2 and 3, respectively, or SEQS ID NO: 4, 5, and 3, respectively; and a light chain comprising a CDRL1, CDRL2, and CDRL3, in which the amino acid sequence of the CDRL1, CDRL2, and CDRL3 is SEQ ID NO: 12, 13 and 14, respectively. The heavy chain may comprise: (a) M253 (EU numbering), in which greater than 6% and no more than 20% of the anti-C5 antibody heavy chains of the composition comprise oxidized M253; and/or (b) M359 (EU numbering), in which greater than 2% and no more than 14% of the anti-C5 antibody heavy chains of the composition comprise oxidized M359; and/or (c) M429 (EU numbering), in which greater than 2% and no more than 14% of the anti-C5 antibody heavy chains of the composition comprise oxidized M429; and/or (d) W33 (EU numbering), in which greater than 1% and no more than 13% of the anti-C5 antibody heavy chains of the composition comprise oxidized W33; and/or (e) W107 (EU numbering), in which greater than 4% and no more than 14% of the anti-C5 antibody heavy chains of the composition comprise oxidized W107 (See Example 1 and Examples 4-5 and Table 10). For any of the pharmaceutical compositions comprising anti-C5 antibody described herein, the heavy chain may comprise (a) and (b); or (a) and (c); or (a) and (d); or (a) and (e); or (b) and (c); or (b) and (d); or (d) and (e); or (c) and (d); or (c) and (e); or (d) and (e). For any of the pharmaceutical compositions comprising anti-C5 antibody described herein, the heavy chain may comprise (a), (b), and (c); or (a), (b), (c), and (d). For some of the pharmaceutical compositions, the heavy chain of the anti-C5 antibody comprises the amino acid sequence of SEQ ID NO: 10 or 11. The light chain may comprise the amino acid sequence of SEQ ID NO: 17. In some embodiments, the pharmaceutical composition comprises a 900 mg anti-C5 antibody dose, such as a unit dose. By way of example, the anti-C5 antibody may be eculizumab.

For some of the pharmaceutical compositions comprising anti-C5 antibody described herein the heavy chain may comprise: (a) M253 (EU numbering), in which greater than 6% and no more than 18% of the anti-C5 antibody heavy chains of the composition comprise oxidized M253; and/or (b) M359 (EU numbering), in which greater than 2% and no more than 4% of the anti-C5 antibody heavy chains of the composition comprise oxidized M359; and/or (c) M429 (EU numbering), in which greater than 2% and no more than 6% of the anti-C5 antibody heavy chains of the composition comprise oxidized M429; and/or (d) W33 (EU numbering), in which greater than 1% and no more than 2% of the anti-C5 antibody heavy chains of the composition comprise oxidized W33; and/or (e) W107 (EU numbering), in which greater than 4% and no more than 11% of the anti-C5 antibody heavy chains of the composition comprise oxidized W107. It is noted that pharmaceutical compositions comprising eculizumab have been determined to have molecular attributes in the aforementioned ranges at a 42-month end-of-shelf timepoint, and have been considered suitable for clinical administration (See Example 1). For any of the pharmaceutical compositions comprising anti-C5 antibody described herein, the heavy chain may comprise (a) and (b); or (a) and (c); or (a) and (d); or (a) and (e); or (b) and (c); or (b) and (d); or (d) and (e); or (c) and (d); or (c) and (e); or (d) and (e). For any of the pharmaceutical compositions comprising anti-C5 antibody described herein, the heavy chain may comprise (a), (b), and (c); or (a), (b), (c), and (d). For some of the pharmaceutical compositions, the heavy chain of the anti-C5 antibody comprises the amino acid sequence of SEQ ID NO: 10 or 11. The light chain may comprise the amino acid sequence of SEQ ID NO: 17. For some of the compositions, the anti-C5 antibody is eculizumab. For some of the pharmaceutical compositions comprising anti-C5 antibody described herein, the heavy chain comprises (a) M253 (EU numbering), in which greater than 6% and no more than 17% of the anti-C5 antibody heavy chains of the composition comprise oxidized M253; and/or (b) M359 (EU numbering), in which greater than 2% and no more than 4% of the anti-C5 antibody heavy chains of the composition comprise oxidized M359; and/or (c) M429 (EU numbering), in which greater than 2% and no more than 6% of the anti-C5 antibody heavy chains of the composition comprise oxidized M429; and/or (d) W33 (EU numbering), in which greater than 1% and no more than 2% of the anti-C5 antibody heavy chains of the composition comprise oxidized W33; and/or (e) W107 (EU numbering), in which greater than 4% and no more than 10% of the anti-C5 antibody heavy chains of the composition comprise oxidized W107. It is noted that pharmaceutical compositions comprising eculizumab have been determined to have molecular attributes in the aforementioned ranges at a 34 to 36-month end-of-shelf timepoint, and have been considered suitable for clinical administration (See Example 1). For any of the pharmaceutical compositions comprising anti-C5 antibody described herein, the heavy chain may comprise (a) and (b); or (a) and (c); or (a) and (d); or (a) and (e); or (b) and (c); or (b) and (d); or (d) and (e); or (c) and (d); or (c) and (e); or (d) and (e). For any of the pharmaceutical compositions comprising anti-C5 antibody described herein, the heavy chain may comprise (a), (b), and (c); or (a), (b), (c), and (d). For some of the pharmaceutical compositions comprising anti-C5 antibody described herein, the heavy chain comprises (a) M253 (EU numbering), in which greater than 6% and no more than 12% of the anti-C5 antibody heavy chains of the composition comprise oxidized M253; and/or (b) M359 (EU numbering), in which greater than 2% and no more than 3% of the anti-C5 antibody heavy chains of the composition comprise oxidized M359; and/or (c) M429 (EU numbering), in which greater than 2% and no more than 5% of the anti-C5 antibody heavy chains of the composition comprise oxidized M429; and/or (d) W33 (EU numbering), in which at least 1% and no more than 2% of the anti-C5 antibody heavy chains of the composition comprise oxidized W33; and/or (e) W107 (EU numbering), in which greater than 4% and no more than 8% of the anti-C5 antibody heavy chains of the composition comprise oxidized W107. For any of the pharmaceutical compositions comprising anti-C5 antibody described herein, the heavy chain may comprise (a) and (b); or (a) and (c); or (a) and (d); or (a) and (e); or (b) and (c); or (b) and (d); or (d) and (e); or (c) and (d); or (c) and (e); or (d) and (e). For any of the pharmaceutical compositions comprising anti-C5 antibody described herein, the heavy chain may comprise (a), (b), and (c); or (a), (b), (c), and (d).

For some of the pharmaceutical compositions comprising anti-C5 antibody described herein, the heavy chain may comprise (a) M253 (EU numbering), in which greater than 7% and no more than 20% of the anti-C5 antibody heavy chains of the composition comprise oxidized M253; and/or (b) M359 (EU numbering), in which greater than 5% and no more than 14% of the anti-C5 antibody heavy chains of the composition comprise oxidized M359; and/or (c) M429 (EU numbering), in which greater than 5% and no more than 14% of the anti-C5 antibody heavy chains of the composition comprise oxidized M429; and/or (d) W33 (EU numbering), in which greater than 2% and no more than 13% of the anti-C5 antibody heavy chains of the composition comprise oxidized W33; and/or (e) W107 (EU numbering), in which greater than 5% and no more than 14% of the anti-C5 antibody heavy chains of the composition comprise oxidized W107. For any of the pharmaceutical compositions comprising anti-C5 antibody described herein, the heavy chain may comprise (a) and (b); or (a) and (c); or (a) and (d); or (a) and (e); or (b) and (c); or (b) and (d); or (d) and (e); or (c) and (d); or (c) and (e); or (d) and (e). For any of the pharmaceutical compositions comprising anti-C5 antibody described herein, the heavy chain may comprise (a), (b), and (c); or (a), (b), (c), and (d). The pharmaceutical composition may comprise a 900 mg anti-C5 antibody dose, such as a unit dose.

For some of the pharmaceutical compositions comprising anti-C5 antibody described herein, the heavy chain comprises (a) M253 (EU numbering), in which greater than 8% and no more than 20% of the anti-C5 antibody heavy chains of the composition comprise oxidized M253; and/or (b) M359 (EU numbering), in which greater than 6% and no more than 14% of the anti-C5 antibody heavy chains of the composition comprise oxidized M359; and/or (c) M429 (EU numbering), in which greater than 6% and no more than 14% of the anti-C5 antibody heavy chains of the composition comprise oxidized M429; and/or (d) W33 (EU numbering), in which greater than 3% and no more than 13% of the anti-C5 antibody heavy chains of the composition comprise oxidized W33; and/or (e) W107 (EU numbering), in which greater than 6% and no more than 14% of the anti-C5 antibody heavy chains of the composition comprise oxidized W107. For any of the pharmaceutical compositions comprising anti-C5 antibody described herein, the heavy chain may comprise (a) and (b); or (a) and (c); or (a) and (d); or (a) and (e); or (b) and (c); or (b) and (d); or (d) and (e); or (c) and (d); or (c) and (e); or (d) and (e). For any of the pharmaceutical compositions comprising anti-C5 antibody described herein, the heavy chain may comprise (a), (b), and (c); or (a), (b), (c), and (d). The pharmaceutical composition may comprise a 900 mg anti-C5 antibody dose, such as a unit dose.

For some of the pharmaceutical compositions comprising anti-C5 antibody described herein, the heavy chain may comprise (a) M253 (EU numbering), wherein greater than 6% and no more than 58% of the anti-C5 antibody heavy chains of the composition comprise oxidized M253; and/or (b) M359 (EU numbering), wherein greater than 2% and no more than 41% of the anti-C5 antibody heavy chains of the composition comprise oxidized M359; and/or (c) M429 (EU numbering), wherein greater than 2% and no more than 41% of the anti-C5 antibody heavy chains of the composition comprise oxidized M429; and/or (d) W33 (EU numbering), wherein greater than 1% and no more than 38% of the anti-C5 antibody heavy chains of the composition comprise oxidized W33; and/or (e) W107 (EU numbering), wherein greater than 4% and no more than 40% of the anti-C5 antibody heavy chains of the composition comprise oxidized W107. For any of the pharmaceutical compositions comprising anti-C5 antibody described herein, the heavy chain may comprise (a) and (b); or (a) and (c); or (a) and (d); or (a) and (e); or (b) and (c); or (b) and (d); or (d) and (e); or (c) and (d); or (c) and (e); or (d) and (e). For any of the pharmaceutical compositions comprising anti-C5 antibody described herein, the heavy chain may comprise (a), (b), and (c); or (a), (b), and (d); or (a), (c), and (d); or (b), (c), and (d); or (a), (b), (c), and (d). The pharmaceutical composition may comprise a 300 mg anti-C5 antibody dose, such as a unit dose.

For some of the pharmaceutical compositions comprising anti-C5 antibody described herein, the heavy chain may comprise (a) M253 (EU numbering), wherein at least 19% and no more than 58% of the anti-C5 antibody heavy chains of the composition comprise oxidized M253; and/or (b) M359 (EU numbering), wherein at least 6% and no more than 41% of the anti-C5 antibody heavy chains of the composition comprise oxidized M359; and/or (c) M429 (EU numbering), wherein at least 5% and no more than 41% of the anti-C5 antibody heavy chains of the composition comprise oxidized M429; and/or (d) W33 (EU numbering), wherein at least 2% and no more than 38% of the anti-C5 antibody heavy chains of the composition comprise oxidized W33; and/or (e) W107 (EU numbering), wherein at least 8% and no more than 40% of the anti-C5 antibody heavy chains of the composition comprise oxidized W107. For any of the pharmaceutical compositions comprising anti-C5 antibody described herein, the heavy chain may comprise (a) and (b); or (a) and (c); or (a) and (d); or (a) and (e); or (b) and (c); or (b) and (d); or (d) and (e); or (c) and (d); or (c) and (e); or (d) and (e). For any of the pharmaceutical compositions comprising anti-C5 antibody described herein, the heavy chain may comprise (a), (b), and (c); or (a), (b), and (d); or (a), (c), and (d); or (b), (c), and (d); or (a), (b), (c), and (d). The pharmaceutical composition may comprise a 300 mg anti-C5 antibody dose, such as a unit dose.

For some of the pharmaceutical compositions comprising anti-C5 antibody described herein, the heavy chain may comprise (a) M253 (EU numbering), wherein greater than 7% and no more than 58% of the anti-C5 antibody heavy chains of the composition comprise oxidized M253; and/or (b) M359 (EU numbering), wherein greater than 5% and no more than 41% of the anti-C5 antibody heavy chains of the composition comprise oxidized M359; and/or (c) M429 (EU numbering), wherein greater than 5% and no more than 41% of the anti-C5 antibody heavy chains of the composition comprise oxidized M429; and/or (d) W33 (EU numbering), wherein greater than 2% and no more than 38% of the anti-C5 antibody heavy chains of the composition comprise oxidized W33; and/or (e) W107 (EU numbering), wherein greater than 5% and no more than 40% of the anti-C5 antibody heavy chains of the composition comprise oxidized W107. For any of the pharmaceutical compositions comprising anti-C5 antibody described herein, the heavy chain may comprise (a) and (b); or (a) and (c); or (a) and (d); or (a) and (e); or (b) and (c); or (b) and (d); or (d) and (e); or (c) and (d); or (c) and (e); or (d) and (e). For any of the pharmaceutical compositions comprising anti-C5 antibody described herein, the heavy chain may comprise (a), (b), and (c); or (a), (b), (c), and (d). The pharmaceutical composition may comprise a 300 mg anti-C5 antibody dose, such as a unit dose.

For some of the pharmaceutical compositions comprising anti-C5 antibody described herein, the heavy chain may comprise (a) M253 (EU numbering), wherein greater than 8% and no more than 58% of the anti-C5 antibody heavy chains of the composition comprise oxidized M253; and/or (b) M359 (EU numbering), wherein greater than 6% and no more than 41% of the anti-C5 antibody heavy chains of the composition comprise oxidized M359; and/or (c) M429 (EU numbering), wherein greater than 6% and no more than 41% of the anti-C5 antibody heavy chains of the composition comprise oxidized M429; and/or (d) W33 (EU numbering), wherein greater than 3% and no more than 38% of the anti-C5 antibody heavy chains of the composition comprise oxidized W33; and/or (e) W107 (EU numbering), wherein greater than 6% and no more than 40% of the anti-C5 antibody heavy chains of the composition comprise oxidized W107. For any of the pharmaceutical compositions comprising anti-C5 antibody described herein, the heavy chain may comprise (a) and (b); or (a) and (c); or (a) and (d); or (a) and (e); or (b) and (c); or (b) and (d); or (d) and (e); or (c) and (d); or (c) and (e); or (d) and (e). For any of the pharmaceutical compositions comprising anti-C5 antibody described herein, the heavy chain may comprise (a), (b), and (c); or (a), (b), (c), and (d). The pharmaceutical composition may comprise a 300 mg anti-C5 antibody dose, such as a unit dose. For any of the pharmaceutical compositions comprising anti-C5 antibody described herein, the heavy chain may comprise (c) M429 (EU numbering), wherein greater than 2% and no more than 6% of the anti-C5 antibody heavy chains of the composition comprise oxidized M429; and/or (e) W107 (EU numbering), wherein greater than 6% and no more than 11% of the anti-C5 antibody heavy chains of the composition comprise oxidized W107.

For any of the pharmaceutical compositions comprising anti-C5 antibody described herein, the anti-C5 antibody may be authorized for administration to a human subject by a government regulatory agency, such as the Food and Drug Administration (FDA) or the European Medicines Agency (EMA). “Authorized for administration to a human subject by a government regulatory agency” refers to for example, an approved Biologics License Application (BLA) by the FDA, or an approved marketing authorization application (MAA) by the EMA.

For any of the pharmaceutical compositions comprising anti-C5 antibody described herein, the percentage of molecular attribute (such as Met+Ox and/or Trp+Ox) may be as determined by reduced peptide mapping as described herein.

It is contemplated that any of the pharmaceutical compositions comprising anti-C5 antibody described herein is suitable for medical use. As such, the pharmaceutical composition comprising anti-C5 antibody is contemplated to have a suitable potency profile. It is noted that residue W107 is in CDRH3 of the anti-C5 antibody having the CDRH3 amino acid sequence of SEQ ID NO: 3 (e.g., in eculizumab). Conventionally, oxidation of residue W107 of such an antibody (e.g., eculizumab) was expected to cause aggregation and/or a loss of potency of the anti-C5 antibody. Unexpectedly, it has been observed herein that the anti-C5 antibody may have W107+Ox levels of 12%, and still maintain at least 80% relative potency, as measured by inhibition of TCC formation (Example 1). Anti-C5 antibody acceptable for medical use at a 42-month end-of-shelf timepoint has been determined to have W107+Ox levels of 11% (Example 2), and as such is also determined to have a clinically acceptable potency. By way of example, the anti-C5 antibody of the pharmaceutical composition may have a relative potency of at least 80%, 81%, 82%, 83%, 84%, 85%, 86%, 87%, 88%, 89%, 90%, 91%, 92%, 93%, 94%, or 95% compared to a reference standard anti-C5 antibody. For example, the reference standard anti-C5 antibody may be from a lot that has been confirmed to inhibit terminal complement complex (TCC) formation. The reference standard anti-C5 antibody may be an anti-C5 antibody having the same amino acid sequences as those of the antibody of the composition. The relative potency of the anti-C5 antibody may comprise or consist of relative inhibition of terminal complement complex (TCC) formation. The relative potency may be as measured by an ELISA assay for determining inhibition of TCC formation as described herein. For example, the relative potency may be as measured by ELISA comprising: immobilizing pre-activated zymosan on a substrate, and subsequently blocking unbound surfaces of the substrate with bovine serum albumin; incubating the anti-C5 antibody with normal human serum comprising C5, so that TCC forms and binds to the zymosan on the substrate; and detecting the TCC with alkaline phosphatase conjugated anti-C5b-9 antibody and an alkaline phosphatase substrate.

For any of the pharmaceutical compositions comprising anti-C5 antibody described herein, the anti-C5 antibody may have a half life suitable for medical use. For example, the anti-C5 antibody may have a half-life of at least 190 hours, such as 190-354 hours. For example, the anti-C5 antibody may have a half-life of about 272 hours. The half-life may be as determined by quantification of free anti-C5 antibody in serum by an electrochemiluminescence (ECL) assay comprising two anti-idiotypic antibodies (one is immobilized on a substrate and one is used for detection), for a dose of anti-C5 antibody of 300 mg-1200 mg or 600 mg-1200 mg intravenously. High mannose molecular attribute has been observed to impact half-life of IgG antibodies. Advantageously, some anti-C5 antibody compositions described herein may maintain a half-life acceptable for medical use across a range of high mannose levels. Based on modeling described herein, some anti-C5 compositions described herein are modeled to tolerate an increase of high mannose species from 0% to 10% without any change of clinical significance for the pharmacokinetic profile (See Example 2). For example, for some anti-C5 antibody compositions described herein an increase of high mannose species from 0% to 10% results in no more than 3.4% (for PNH patients) and 4.5% (for aHUS patients) increase in clearance of anti-C5 antibody species. Levels of high mannose can be determined by peptide mapping comprising trypsin digest and analysis by liquid chromatography (LC)-mass spectrometry (MS)/MS, for example as described in Goetze, et al., Glycobiology 21:949-959 (2011), or by HILIC (hydrophilic interaction liquid chromatography) glycan mapping. Alternatively, in some embodiments, the half-life may be as determined by quantification of total (bound+free) anti-C5 antibody in serum by ELISA comprising human C5 immobilized on a substrate.

Any of the pharmaceutical compositions described herein may further comprise additional components. For example, some of the pharmaceutical compositions described herein may comprise a buffer. The buffer may comprise acetate, optionally between 5 mM and 20 mM. Some of the pharmaceutical compositions described herein may comprise a stabilizer. The stabilizer may comprise sorbitol, optionally about 5% (w/v). Some of the pharmaceutical compositions described herein may comprise a chelating agent. The chelating agent may comprise ethylenediaminetetraacetic acid (EDTA), optionally at a concentration of about 0.01 mM to about 0.05 mM, for example 0.05±25% mM. Some of the pharmaceutical compositions described herein may comprise between 5 mM and 20 mM acetate buffer; about 5% (w/v) sorbitol; and about 0.01 mM to about 0.05 mM EDTA. Some of the pharmaceutical compositions described herein may comprise between 5 mM and 20 mM acetate buffer; about 5% (w/v) sorbitol; and about 0.038 mM to about 0.063 mM EDTA. It is contemplated that formulating the anti-C5 antibody in pharmaceutical compositions comprising a chelating agent such as EDTA can limit the formation of oxidized species such as W107+Ox.

For any of the pharmaceutical compositions comprising anti-C5 antibody described herein, the composition may comprise or consist of a 300 mg-900 mg dose of the anti-C5 antibody, such as a unit dose thereof. For any of the pharmaceutical compositions comprising anti-C5 antibody described herein, the composition may comprise or consist of a 300 mg-1200 mg dose of the anti-C5 antibody, such as a unit dose thereof. For any of the pharmaceutical compositions comprising anti-C5 antibody described herein, the composition may comprise or consist of a 300 mg dose of the anti-C5 antibody, such as a unit dose thereof. For any of the pharmaceutical compositions comprising anti-C5 antibody described herein, the composition may comprise or consist of 900 mg dose of the anti-C5 antibody, such as a unit dose thereof.

### Methods Comprising Administering the Pharmaceutical Composition

Any of the pharmaceutical compositions described herein may be for medical use. Described herein are methods comprising administering a pharmaceutical composition as described herein to a subject in need of treatment with an anti-C5 antibody. The subject may be a human. The administration may be intravenous. In some methods, the subject may have myasthenia gravis, paroxysmal nocturnal hemoglobinuria, neuromyelitis optica spectrum disorder, or atypical hemolytic uremic syndrome. In some methods, the anti-C5 antibody is administered to the subject at a dose of 300 mg-1200 mg or 600 mg-1200 mg, for example 300 mg, 600 mg, 900 mg, or 1200 mg. For example, the anti-C5 antibody may be administered at a dose of: (a) 600 mg weekly for 4 weeks, followed by a 900 mg dose 1 week later, followed by 900 mg every 2 weeks thereafter; or (b) 900 mg weekly for 4 weeks, followed by a 1200 mg dose 1 week later, followed by 1200 mg every 2 weeks thereafter. For example, for any of the pharmaceutical compositions described herein, the anti-C5 antibody may be administered at a dose of 300 mg. For example, for any of the pharmaceutical compositions described herein, the anti-C5 antibody may be administered at a dose of 900 mg. By way of example, the anti-C5 antibody may be eculizumab.

### Methods of Manufacturing Pharmaceutical Composition

Described herein are methods of manufacturing a pharmaceutical composition comprising anti-C5 antibody as described herein. Methods of manufacturing are contemplated in which anti-C5 antibodies of the pharmaceutical composition have Trp+Ox and/or Met+Ox levels within the ranges taught herein. The method may comprise providing a composition comprising the anti-C5 antibody, for example a drug substance or a drug product. The method may comprise determining the percentage of heavy chains of the anti-C5 antibody of the composition that comprise (a) M253 (EU numbering) oxidation; (b) M359 (EU numbering) oxidation; (c) M429 (EU numbering) oxidation; (d) W33 (EU numbering) oxidation; and/or (e) W107 (EU numbering) oxidation. The method may comprise manufacturing the composition for pharmaceutical use if the heavy chains comprise: (a) M253 (EU numbering), in which greater than 6% and no more than 20% of the anti-C5 antibody heavy chains of the composition comprise oxidized M253; and/or (b) M359 (EU numbering), in which greater than 2% and no more than 14% of the anti-C5 antibody heavy chains of the composition comprise oxidized M359; and/or (c) M429 (EU numbering), in which greater than 2% and no more than 14% of the anti-C5 antibody heavy chains of the composition comprise oxidized M429; and/or (d) W33 (EU numbering), in which greater than 1% and no more than 13% of the anti-C5 antibody heavy chains of the composition comprise oxidized W33; and/or (e) W107 (EU numbering), in which greater than 4% and no more than 14% of the anti-C5 antibody heavy chains of the composition comprise oxidized W107. The method may comprise manufacturing the composition for pharmaceutical use if the heavy chains comprise: (a) M253 (EU numbering), in which greater than 6% and no more than 18% of the anti-C5 antibody heavy chains of the composition comprise oxidized M253; and/or (b) M359 (EU numbering), in which greater than 2% and no more than 4% of the anti-C5 antibody heavy chains of the composition comprise oxidized M359; and/or (c) M429 (EU numbering), in which greater than 2% and no more than 6% of the anti-C5 antibody heavy chains of the composition comprise oxidized M429; and/or (d) W33 (EU numbering), in which greater than 1% and no more than 2% of the anti-C5 antibody heavy chains of the composition comprise oxidized W33; and/or (e) W107 (EU numbering), in which greater than 4% and no more than 11% of the anti-C5 antibody heavy chains of the composition comprise oxidized W107. “Manufacturing the composition for pharmaceutical use” refers to additional processing to place the pharmaceutical composition in a suitable condition to be released for medical use. For example, manufacturing the composition for pharmaceutical use may comprise formulating the anti-C5 antibody of the composition in formulation buffer, such as acetate. For example, manufacturing the composition for pharmaceutical use may comprise filling a container, such as a vial, syringe, or autoinjector with the pharmaceutical composition. On the other hand, if the heavy chains of the of the anti-C5 antibody of the composition do not comprise M253 oxidation, M359 oxidation, M429 oxidation, W33 oxidation, and/or W107 oxidation within the specified range(s), the pharmaceutical composition may not be manufactured. Optionally, such a pharmaceutical composition comprising molecular attributes outside of the specified range(s) may instead be discarded. It will be appreciated that manufacturing the composition for pharmaceutical use does not necessarily require that a pharmaceutical composition contain the same molecule of antibody that was determined to have a particular molecular attribute or potency. Rather, a sample may be tested from a production lot, and if the sample indicates that the production lot is suitable for manufacture for pharmaceutical use, that lot may be manufactured for pharmaceutical use. In some methods, the composition is manufactured for pharmaceutical use if the heavy chain comprises: (a) and (b); or (a) and (c); or (a) and (d); or (a) and (e); or (b) and (c); or (b) and (d); or (d) and (e); or (c) and (d); or (c) and (e); or (d) and (e). In some methods, the composition is manufactured for pharmaceutical use if the heavy chain comprises (a), (b), and (c); or (a), (b), (c), and (d). By way of example, the anti-C5 antibody may be eculizumab.

In some methods, the composition is manufactured for pharmaceutical use if the heavy chain comprises: (a) M253 (EU numbering), in which greater than 6% and no more than 17% of the anti-C5 antibody heavy chains of the composition comprise oxidized M253; and/or (b) M359 (EU numbering), in which greater than 2% and no more than 4% of the anti-C5 antibody heavy chains of the composition comprise oxidized M359; and/or (c) M429 (EU numbering), in which greater than 2% and no more than 6% of the anti-C5 antibody heavy chains of the composition comprise oxidized M429; and/or (d) W33 (EU numbering), in which greater than 1% and no more than 2% of the anti-C5 antibody heavy chains of the composition comprise oxidized W33; and/or (e) W107 (EU numbering), in which greater than 4% and no more than 10% of the anti-C5 antibody heavy chains of the composition comprise oxidized W107.

In some methods, the composition is manufactured for pharmaceutical use if the heavy chain comprises: (a) M253 (EU numbering), wherein greater than 6% and no more than 12% of the anti-C5 antibody heavy chains of the composition comprise oxidized M253; and/or (b) M359 (EU numbering), wherein greater than 2% and no more than 3% of the anti-C5 antibody heavy chains of the composition comprise oxidized M359; and/or (c) M429 (EU numbering), wherein greater than 2% and no more than 5% of the anti-C5 antibody heavy chains of the composition comprise oxidized M429; and/or (d) W33 (EU numbering), wherein at least 1% and no more than 2% of the anti-C5 antibody heavy chains of the composition comprise oxidized W33; and/or (e) W107 (EU numbering), wherein greater than 4% and no more than 8% of the anti-C5 antibody heavy chains of the composition comprise oxidized W107. In some methods, the composition is manufactured for pharmaceutical use if the heavy chain comprises: (c) M429 (EU numbering), wherein greater than 2% and no more than 6% of the anti-C5 antibody heavy chains of the composition comprise oxidized M429; and/or (e) W107 (EU numbering), wherein greater than 6% and no more than 11% of the anti-C5 antibody heavy chains of the composition comprise oxidized W107.

In some methods, the composition is manufactured for pharmaceutical use if the heavy chain comprises: (a) M253 (EU numbering), wherein greater than 7% and no more than 18% of the anti-C5 antibody heavy chains of the composition comprise oxidized M253; and/or (b) M359 (EU numbering), wherein greater than 3% and no more than 4% of the anti-C5 antibody heavy chains of the composition comprise oxidized M359; and/or (c) M429 (EU numbering), wherein greater than 3% and no more than 6% of the anti-C5 antibody heavy chains of the composition comprise oxidized M429; and/or (d) W33 (EU numbering), wherein greater than 1% and no more than 2% of the anti-C5 antibody heavy chains of the composition comprise oxidized W33; and/or (e) W107 (EU numbering), wherein greater than 5% and no more than 11% of the anti-C5 antibody heavy chains of the composition comprise oxidized W107.

In some methods, the composition is manufactured for pharmaceutical use if the heavy chain comprises: (a) M253 (EU numbering), wherein greater than 8% and no more than 18% of the anti-C5 antibody heavy chains of the composition comprise oxidized M253; and/or (b) M359 (EU numbering), wherein greater than 3% and no more than 4% of the anti-C5 antibody heavy chains of the composition comprise oxidized M359; and/or (c) M429 (EU numbering), wherein greater than 4% and no more than 6% of the anti-C5 antibody heavy chains of the composition comprise oxidized M429; and/or (d) W33 (EU numbering), wherein greater than 1% and no more than 2% of the anti-C5 antibody heavy chains of the composition comprise oxidized W33; and/or (e) W107 (EU numbering), wherein greater than 6% and no more than 11% of the anti-C5 antibody heavy chains of the composition comprise oxidized W107.

In some methods, the composition is manufactured for pharmaceutical use if the heavy chain comprises: (a) M253 (EU numbering), wherein greater than 6% and no more than 58% of the anti-C5 antibody heavy chains of the composition comprise oxidized M253; and/or (b) M359 (EU numbering), wherein greater than 2% and no more than 41% of the anti-C5 antibody heavy chains of the composition comprise oxidized M359; and/or (c) M429 (EU numbering), wherein greater than 2% and no more than 41% of the anti-C5 antibody heavy chains of the composition comprise oxidized M429; and/or (d) W33 (EU numbering), wherein greater than 1% and no more than 38% of the anti-C5 antibody heavy chains of the composition comprise oxidized W33; and/or (e) W107 (EU numbering), wherein greater than 4% and no more than 40% of the anti-C5 antibody heavy chains of the composition comprise oxidized W107. The composition may be manufactured for a 300 mg anti-C5 antibody dose.

In some methods, the composition is manufactured for pharmaceutical use if the heavy chain comprises: (a) M253 (EU numbering), wherein greater than 7% and no more than 58% of the anti-C5 antibody heavy chains of the composition comprise oxidized M253; and/or (b) M359 (EU numbering), wherein greater than 5% and no more than 41% of the anti-C5 antibody heavy chains of the composition comprise oxidized M359; and/or (c) M429 (EU numbering), wherein greater than 5% and no more than 41% of the anti-C5 antibody heavy chains of the composition comprise oxidized M429; and/or (d) W33 (EU numbering), wherein greater than 2% and no more than 38% of the anti-C5 antibody heavy chains of the composition comprise oxidized W33; and/or (e) W107 (EU numbering), wherein greater than 5% and no more than 40% of the anti-C5 antibody heavy chains of the composition comprise oxidized W107. The composition may be manufactured for a 300 mg anti-C5 antibody dose.

In some methods, the composition is manufactured for pharmaceutical use if the heavy chain comprises: (a) M253 (EU numbering), wherein at least 19% and no more than 58% of the anti-C5 antibody heavy chains of the composition comprise oxidized M253; and/or (b) M359 (EU numbering), wherein at least 6% and no more than 41% of the anti-C5 antibody heavy chains of the composition comprise oxidized M359; and/or (c) M429 (EU numbering), wherein at least 2% and no more than 41% of the anti-C5 antibody heavy chains of the composition comprise oxidized M429; and/or (d) W33 (EU numbering), wherein at least 1% and no more than 38% of the anti-C5 antibody heavy chains of the composition comprise oxidized W33; and/or (e) W107 (EU numbering), wherein at least 8% and no more than 40% of the anti-C5 antibody heavy chains of the composition comprise oxidized W107. The composition may be manufactured for a 300 mg anti-C5 antibody dose.

In some methods, the composition is manufactured for pharmaceutical use if the composition comprises no more than 7.0% HMW species of anti-C5 antibody as determined by SE-UHPLC. For example, the composition may be manufactured for pharmaceutical use if the composition comprises greater than 1.5% HMW species of anti-C5 antibody, and no more than 7.0% HMW species of anti-C5 antibody as determined by SE-UHPLC. For example, the composition may be manufactured for pharmaceutical use if the composition comprises greater than 2% HMW species of anti-C5 antibody, and no more than 7.0% HMW species of anti-C5 antibody as determined by SE-UHPLC. In some methods, the composition is manufactured for pharmaceutical use if the composition comprises no more than 5.0% HMW species of anti-C5 antibody as determined by SE-UHPLC. For example, the composition may be manufactured for pharmaceutical use if the composition comprises greater than 1.5% HMW species of anti-C5 antibody, and no more than 5.0% HMW species of anti-C5 antibody as determined by SE-UHPLC. For example, the composition may be manufactured for pharmaceutical use if the composition comprises at least 1.7% HMW species of anti-C5 antibody, and no more than 5.0% HMW species of anti-C5 antibody as determined by SE-UHPLC. For example, the composition may be manufactured for pharmaceutical use if the composition comprises greater than 1.8% HMW species of anti-C5 antibody, and no more than 5.0% HMW species of anti-C5 antibody as determined by SE-UHPLC. (a) M253 (EU numbering), wherein greater than 8% and no more than 58% of the anti-C5 antibody heavy chains of the composition comprise oxidized M253; and/or (b) M359 (EU numbering), wherein greater than 6% and no more than 41% of the anti-C5 antibody heavy chains of the composition comprise oxidized M359; and/or (c) M429 (EU numbering), wherein greater than 6% and no more than 41% of the anti-C5 antibody heavy chains of the composition comprise oxidized M429; and/or (d) W33 (EU numbering), wherein greater than 3% and no more than 38% of the anti-C5 antibody heavy chains of the composition comprise oxidized W33; and/or (e) W107 (EU numbering), wherein greater than 6% and no more than 40% of the anti-C5 antibody heavy chains of the composition comprise oxidized W107. The composition may be manufactured for a 300 mg anti-C5 antibody dose.

In some methods, the composition is manufactured for pharmaceutical use if the composition comprises no more than 2.0% HMW species of anti-C5 antibody as determined by SE-UHPLC. For example, the composition may be manufactured for pharmaceutical use if the composition comprises greater than 1.5% HMW species of anti-C5 antibody, and no more than 2.0% HMW species of anti-C5 antibody as determined by SE-UHPLC. For example, the composition may be manufactured for pharmaceutical use if the composition comprises greater than 1.6% HMW species of anti-C5 antibody, and no more than 2.0% HMW species of anti-C5 antibody as determined by SE-UHPLC. For example, the composition may be manufactured for pharmaceutical use if the composition comprises at least 1.7% HMW species of anti-C5 antibody, and no more than 2.0% HMW species of anti-C5 antibody as determined by SE-UHPLC. For example, the composition may be manufactured for pharmaceutical use if the composition comprises at least 1.8% HMW species of anti-C5 antibody, and no more than 2.0% HMW species of anti-C5 antibody as determined by SE-UHPLC.

In some methods, the composition is manufactured for pharmaceutical use only if the recited molecular attributes are within the specified range.

### EXAMPLES

### Example 1: Molecular Attributes of Eculizumab

### Reduced Peptide Mapping

Molecular attributes of eculizumab (an anti-C5 monoclonal antibody) were assessed by reduced peptide mapping. In brief, sample was reduced with dithiothreitol (DTT) in denaturant and alkylated with iodoacetic acid (IAA). Excess reagents were removed by size exclusion-based desalting columns. Digestion with trypsin was carried out at 37° C. for 1 h. Peptides from the digest were separated by RP-UPLC in a trifluoroacetic acid/acetonitrile (TFA/ACN) gradient with on-line MS and MS/MS data collection. The sample preparation conditions described for reduction and alkylation are appropriate for 500 μg total protein.

Materials used included:
Digestion buffer: 50 mM Tris HCl pH 7.5
Denaturing buffer: 7.5 M Guanidine HCl, 0.25 M Tris HCl, 2 mM EDTA pH 7.5
0.5 M Dithiothreitol (DTT) in denaturing buffer
0.5 M Iodoacetic Acid (IAA) in denaturing buffer

Eculizumab was added to denaturing buffer to a concentration of 1 mg/ml eculizumab in 500 μL. To reduce the eculizumab, 9 μL of 0.5 M DTT was added to the eculizumab (to a final concentration of about 9 mM DTT), and the mixture was incubated at 27° C. for 23 minutes. The reduced, denatured eculizumab was then alkylated by adding 17 μL of 0.5 M IAA (to a concentration of about 16 mM IAA) and incubating at 27° C. for 19 minutes. The eculizumab was then desalted using a NAP-5 column. The column was equilibrated in around 2.5 mL of 50 mM Tris-HCl pH 7.5, followed by three times with around 2.5 ml of 50 mM Tris-HCl pH 7.5. Then, 500 μL of reduced and alkylated eculizumab sample was added to the equilibrated NAP-5 column, and desalted eculizumab sample was collected. 9.0 μl of 1 mg/ml trypsin was added to a volume of 100 μl of desalted eculizumab (to a concentration of about 0.08 mg/ml trypsin), and the eculizumab was digested at 37° C. for 1 hour. The digest was then quenched by adding 6 μL of 10% TFA to the eculizumab (so that the sample was quenched in about 0.5% TFA).

RP-UPLC was performed using a Waters BEH C4 Column, 300 Å, 1.7 μm, 1 mm×150 mm on a Waters ACQUITY UPLC apparatus using the following parameters:

TABLE 1

| Parameter | Conditions | |
|---|---|---|
| Mobile Phase A | 0.1% Trifluoracetic acid | |
| Mobile Phase B | 0.1% TFA in Acetonitrile | |
| Column | Waters BEH C4 Column 1.7 μm, | |
| | 2.1 × 150 mm | |
| Column Temperature | 50° | C. |
| Sample Tray Temp. | 2-8° | C. |
| Flow Rate | 200 | μL/minute |
| Injection | 50 | μL |

| | |
|---|---|
| Gradient | From 0.5% to 45% B from 3 to 93 |
| | minutes at 50° C. |

The UPLC gradients were either as shown in Table 2.1 or 2.2, and produced comparable resolution:

TABLE 2.1

| Time (min) | FlowRate (ml/min) | B % |
|---|---|---|
| 0.00 | 0.2 | 0.5 |
| 3.00 | 0.2 | 0.5 |
| 93 | 0.2 | 45 |
| 94 | 0.4 | 100 |
| 97 | 0.4 | 100 |
| 98 | 0.4 | 0.5 |
| 108 | 0.4 | 0.5 |
| 118 | 0.2 | 0.5 |

TABLE 2.2

| Time (min) | Flow rate (ml/min) | B % |
|---|---|---|
| 0.00 | 0.2 | 0.5 |
| 3.00 | 0.2 | 0.5 |
| 93 | 0.2 | 45 |
| 94 | 0.4 | 100 |
| 97 | 0.4 | 100 |
| 98 | 0.4 | 0.5 |
| 108 | 0.4 | 0.5 |
| 118 | 0.2 | 0.5 |

The sample was then analyzed by mass spectrometry on a Thermo Orbitrap instrument. Most data were collected on Thermo Q Exactive/Q Exactive Plus Orbitrap instrument, though some data were collected on an earlier version of the instrument, a Thermo Orbitrap Elite instrument. The data were analyzed for molecular attributes, including Methionine Oxidation (Met+Ox) and Tryptophan Oxidation (Trp+Ox). Residue positions from the analysis are on the eculizumab heavy chain, identified by EU numbering. Parameters for the mass spectrometer instrument are shown in Table 3 below:

TABLE 3
Parameters for Thermo Q Exactive/Q Exactive
Plus Orbitrap Mass Spectrometer

| Parameter | Conditions | |
|---|---|---|
| Instrument | Thermo Q Exactive/Q Exactive Plus | |
| | Orbitrap or equivalent | |
| Divert Valve | Divert to waste from 0-5 min; | |
| | Divert to sample collection from | |
| | 5 to 100 min; | |
| | Divert to waste from 100 min to | |
| | 118 min. | |
| MS collection Run Time | 4 to 94 | min |
| Polarity | Positive | |
| Scan Range | 300 to 1800 | m/z |
| Full MS Resolution | 70,000 | |
| Full MS AGC Target | 1e6 | |
| Full MS Maximum IT | 200 | ms |
| MS2 Resolution | 17,500 | |
| MS2 AGC target | 5e5 | |
| MS2 Maximum IT | 200 | ms |
| MS2 Loop count | 4 | |
| MS2 Isolation window | 3.0 | m/z |
| MS2 (N)CE/stepped nce | 30 | |
| Minimum AGC target | 5e3 | |
| Charge exclusion | Unassigned, 6-8, >8 | |
| Exclude isotope | on | |
| Dynamic exclusion | 10 | s |

Mass spectrometry data were processed using MassAnalyzer software versions 4.02-4.13 (Z. Zhang, Large-Scale Identification and Quantification of Covalent Modifications in Therapeutic Proteins. Anal. Chem. (2009), 81 (20), 8354-8364). Parameters in Data Processing Options were set with S/N Threshold between 20-100, and Mass Accuracy (ppm) at 5. Data were exported to Microsoft EXCEL with default parameters (Peptide Selection Criteria: 17% of the most abundant peptide; Charge Selection Criteria: 33% of the most abundant charge state). Percent values of attributes were calculated as follows:

TABLE 4

| | Modifications Identified in | |
|---|---|---|
| Modification | MassAnalyzer | Reporting |
| Methionine (M) Oxidation | Oxidation | Sum of all types of oxidation detected on |
| | Double Oxidation | the same methionine (M) residue. |
| Tryptophan (W) Oxidations | Oxidation | Sum of all types of oxidation detected on |
| | Double Oxidation | the same tryptophan (W) residue. |
| | Double Oxidation - H2O | |
| | Triple Oxidation | |
| | Oxidation to hydroxy- | |
| | kynurenine | |
| | Oxidation to kynurenine | |
| Deamidation | Deamination | Sum of deamidation and NH3 loss |
| | NH3 loss | detected on the same Asparagine (N)/ |
| | | Glutamine (Q) residue. For N-terminal |
| | | N/Q, directly report deamidation |
| | | reported in MassAnalyzer. |
| Isomerization | Isomerization | Sum of deamidation and NH3 loss |
| | H20 loss | detected on the same Glutamic Acid (E)/ |
| | | Aspartic Acid (D) residue. For N-terminal |
| | | E/D, directly report Isomerization |
| | | reported in MassAnalyzer. |

Relative Abundance of Modification = Peak Area of Modified Peptides/ (Peak Area of Modified Peptides + Peak Area of Unmodified peptides. Peak area information can be exported from MassAnalyzer. It is noted that in some MassAnalyzer versions prior to 4.06, W double oxidation - H₂O was identified as +14 Da modification. Accordingly, to perform these calculations using these earlier MassAnalyzer versions, W double oxidation - H₂O should be accounted for.

Potency of sample at different timepoints was measured by an enzyme-linked immunosorbent assay (ELISA) that measures the ability of eculizumab to prevent the formation of the terminal complement complex (TCC). The details of the ELISA are described in this Example.

Samples of clinical eculizumab formulation, as well as Soliris® reference product (“reference product”) of various ages were analyzed. In the examples herein, unless stated otherwise “reference product” shall refer to Soliris® reference product, and “eculizumab composition” or simply “eculizumab” will refer to eculizumab compositions according to embodiments herein. Lot numbers 1, 2, and 3 were formulated as 10 mg/ml eculizumab in 10 mM acetate, 5% (w/v) sorbitol, 0.01% polysorbate 80, pH 5.2. The molecular attribute and relative potency values for the analyzed samples are shown in Table 5 (eculizumab compositions) and Table 7 (reference product).

TABLE 5
Eculizumab molecular attributes

| Lot | Met 253 + | Met 359 + | Met 429 + | Trp 33 + Ox | Trp 107 + Ox | Rel. | Age |
|---|---|---|---|---|---|---|---|
| Number | Ox (%) | Ox (%) | Ox (%) | (sum) (%) | (sum) (%) | Potency | (months) |
| 1 | 2% | 1% | 1% | <1% | 1% | 114% | 0 |
| 2 | 2% | 1% | 1% | <1% | 1% | 114% | 0 |
| 3 | 2% | 1% | 1% | <1% | 1% | 115% | 0 |
| 1 | 4% | 1% | 1% | 1% | 4% | 105% | 18 |
| 2 | 3% | 1% | 1% | <1% | 3% | N/A | 16 |
| 1 | 17% | 4% | 6% | 2% | 9% | 92% | 36 |
| 2 | 14% | 4% | 5% | 2% | 10% | 81% | 34 |
| | | | | | | (36 | |
| | | | | | | Month)* | |
| 3 | 12% | 3% | 5% | 2% | 8% | 85% | 34 |
| | | | | | | (36 | |
| | | | | | | Month)* | |
| 1 | 23% | 5% | 8% | 3% | 13% | 76% | 48 |
| 2 | 22% | 5% | 8% | 3% | 14% | 68% | 48 |
| 3 | 20% | 5% | 7% | 3% | 12% | 80% | 48 |

*This row shows values for molecular attributes calculated at 34 months, and relative potency for the same lot calculated at 36 months. It is contemplated that the relative potency at 34 months would be no lower than the relative potency at 36 months.

Accordingly, oxidation of Met253, Met359, Met429, Trp33, and Trp107 on the heavy chain was detected in the eculizumab formulations.

Formulations of eculizumab up to 42 months old were considered to have an acceptable potency and tolerability profile for clinical use. Based on linear regression analysis of the data shown in Table 5, the attribute levels of each of the following attributes at 42 months was interpolated as shown in Table 6:

TABLE 6

| Molecular | (%) Molecular Attribute | | |
|---|---|---|---|
| attribute | at 42 months | Linear regression equation | R² |
| Met 253 + Ox | 18 | y = 0.4217x + 0.0776 | R² = 0.9198 |
| Met 359 + Ox | 4 | y = 0.0931x + 0.4162 | R² = 0.9032 |
| Met 429 + Ox | 6 | y = 0.1434x + 0.2781 | R² = 0.9263 |
| Trp 33 + Ox | 2 | y = 0.0573x − 0.0574 | R² = 0.9072 |
| Trp 107 + Ox | 11 | y = 0.2578x + 0.2255 | R² = 0.9746 |

Accordingly, it is contemplated that that eculizumab compositions having molecular attribute levels at least as high as those shown in Table 6 (42 months) are considered to have a suitable profile for clinical use.

For reference, molecular attribute levels of reference product were also determined using the described reduced peptide mapping technique:

TABLE 7
Reference product molecular attributes**

| | Met 253 + | Met 359 + Ox | Met 429 + Ox | Trp 33 + Ox | Trp 107 + Ox | Age |
|---|---|---|---|---|---|---|
| Lot Number | Ox (%) | (%) | (%) | (sum) (%) | (sum) (%) | (months) |
| A | 4% | 1% | 2% | <1% | 4% | 30* |
| C | 4% | 1% | 1% | <1% | 3% | 27 |
| J | 4% | 1% | 1% | 1% | 2% | 26 |
| B | 4% | 1% | 1% | <1% | 4% | 25 |
| E | 4% | 1% | 1% | N/D | 1% | 24 |
| H | 4% | 1% | 1% | N/D | 1% | 24 |
| L | 5% | 1% | 1% | 1% | 1% | 24 |
| I | 5% | 1% | 1% | N/D | 1% | 23 |
| M | 6% | 1% | 2% | <1% | 2% | 22 |
| K | 4% | 2% | 1% | <1% | 1% | 21 |
| F | 4% | 1% | 1% | N/D | 1% | 20 |
| O | 4% | 1% | 1% | <1% | 1% | 20 |
| D | 6% | 2% | 2% | 1% | 4% | 19 |
| A | 3% | 1% | 1% | N/D | 1% | 17 |
| G | 3% | 1% | 1% | N/D | 1% | 16 |
| N | 3% | 1% | 1% | <1% | 1% | 16 |

*Reference product expiry is identified by manufacturer as 30 months.
**% abundance >0 and <0.5% is reported as <1%; % abundance >/+0.5% is reported as 1%; % abundance >1% are rounded to closet integer.

### Anti-C5 Antibody Potency Analysis

To assess potency of the anti-C5 antibody, an ELISA that measures the ability of anti-C5 antibody to prevent the formation of the TCC was performed as follows:

Microtiter plates are coated with zymosan (5 μg/mL solution for at least 16 hours at 2-8° C.), which acts as a potent complement activator. Microtiter plates are then blocked with a 1% bovine serum albumin solution, and incubated at room temperature for 1-4 hours. Varying concentrations, forming a dose curve, of anti-C5 antibody reference standard, anti-C5 antibody control sample, and anti-C5 antibody test samples are added to a fixed concentration of normal human serum (NHS) and added to the microtiter plates and incubated for 50-60 minutes. The NHS acts as a source of complement proteins. Positive controls containing no anti-C5 antibody, and negative controls containing C5-depleted human serum (C5 DHS) are also added to the microtiter plates. During incubation, NHS complement proteins are activated by the zymosan coating on the microtiter plates. The TCC, generated as a result of terminal complement activation, binds to the zymosan and is then detected with an alkaline phosphatase conjugated anti-C5b-9 antibody. The addition of an alkaline phosphatase substrate initiates a colorimetric reaction that is subsequently stopped using an acidic solution. The alkaline phosphatase conjugated anti-C5b-9 antibody is incubated at room temperature for 25-35 minutes, and the reaction with the alkaline phosphatase substrate solution is performed at room temperature for 30-40 minutes. Once the reaction is stopped the absorbance is read at 405 nm. The amount of complement activation correlates with the absorbance at 405 nm. This assay measures the anti-C5 antibody dose dependent decrease of TCC formation. Anti-C5 antibody test sample activity is determined by comparing the test sample response to the response obtained with the anti-C5 antibody reference standard. The data from the dose curves (anti-C5 antibody reference standard, the anti-C5 antibody control, and the anti-C5 antibody test samples) are modeled using a 4-parameter logistic curve fit. Using parallel line analysis, the EC50 of the dose response curve from the anti-C5 antibody control and test samples are compared to the EC50 of the anti-C5 antibody reference standard. This comparison generates a relative potency ratio which can be expressed as percent relative potency.

### Example 2: Analysis of High Mannose Glycans

It has been observed that IgG of the high mannose (HM) glycoform appears to be cleared faster. Without being limited by theory, it is contemplated that the clearance mechanism is likely to involve the binding of high mannose to the mannose receptor. Eculizumab is an IgG2/IgG4 hybrid molecule. However, the structures of high mannose glycans are the same in IgG1, IG2 and IgG4 molecules and are expected to bind to the mannose receptor similarly.

A modeling approach was used to estimate the potential impact of a change in % HM on the clearance of eculizumab. The model assumed a half-life of 11.3 days (for a PNH patient) and 17.3 days (for an aHUS patient), and an increased rate constant of 0.035 Day-1 for the HM form of eculizumab. The increased rate constant was based on the HM % decrease rate of a reference IgG2 antibody following a single IV dose (this was a conservative estimate based on the highest observed change rate for a reference IgG2 molecule). The PK profile was modeled up to 56.5 days (for a PNH patient) or 86.3 days (for aHUS patient), and the duration equivalent to 5 half-lives with no correction for preferential pairing. The potential impact was estimated for a change in % HM from 0% to 2%, 4%, 6%, 8% and 10%.

For a half-life of 11.3 days (PNH patient) an increase in % HM from 0% to 10% was modeled to result in a maximum estimated 3.4% increase in the clearance of eculizumab up to 56.5 days (5 half-lives), and a corresponding 3.4% decrease in eculizumab exposure (AUC) up to 56.5 days (5 half-lives).

For a half-life of 17.3 days (aHUS patient), an increase in % HM from 0% to 10% was modeled to result in a maximum estimated 4.5% increase in the clearance of eculizumab up to 86.3 days (5 half-lives), and a corresponding 4.5% decrease in eculizumab exposure (AUC) up to 86.3 days (5 half-lives).

The modelling results for both half-lives (11.3 days and 17.3 days) suggest that a change in the % HM of eculizumab from 0% to 10% would cause only a minimal change in AUC of eculizumab that would not be of practical significance for the PK profile.

### Example 3: Analysis of High Molecular Weight Species

### Analysis of Clinical Impact of HMW Species

High molecular weight (HMW) species of eculizumab in clinical pharmaceutical compositions after different storage durations at 5° C. were determined by SE-UHPLC. From these data, stability profiles modeling the rate of HMW species formation over time at 5° C. were developed. Based on these linear regression profiles, the HMW species exposure was calculated for lots suitable for medical use at the time of clinical administration (See International Patent App. No. PCT/US21/63641). Data were calculated for lots used in a Phase 1 clinical trial:

TABLE 8

| Trial # | Linear Regression Equation | R² |
|---|---|---|
| 1 | y = 0.0122x + 0.428 | 0.9792 |
| 2 | y = 0.0132x + 0.425 | 0.9695 |

“y” refers to % HMW species at the end of storage duration; “x” refers to storage duration (months) at 5° C.; y-intercepts refer to % HMW species prior to the storage.

It was calculated that pharmaceutical compositions comprising eculizumab for clinical administration comprised up to 1.82 mg HMW species at the time of administration in clinical studies. 1.82 mg HMW species corresponds to 0.60% in a 300 mg dose, or 0.20% in a 900 mg dose of eculizumab. Data were based on clinical administration of the 900 mg dose of eculizumab, and the calculated percent HMW species value for a 300 mg dose is based on the relative mass of a 900 mg eculizumab dose compared to a 300 mg eculizumab dose. Adverse Events (AE) analyzed included occurrence of ADA (anti-drug antibody), infection, infusion reaction, headache, fatigue. No AE was observed to positively correlate with exposures to the HMW species in a Phase 1 clinical study.

Using data from a Phase 3 clinical trial, it was calculated that pharmaceutical compositions comprising eculizumab or reference product for clinical administration comprised up to 14.79 mg HMW species at the time of clinical administration. 14.79 mg HMW species corresponds to 4.93% in a 300 mg dose, or 1.64% in a 900 mg dose of eculizumab. It was calculated that reference product contained up to 1.64% HMW species at the time of administration in a 900 mg dose. For clarity, patients were dosed in the Phase 3 study at 900 mg and the 300 mg dose was calculated based on the relative gravimetric amount of attribute exposure. AE analyzed included infection, headache, anemia, vaccination, pyrexia, cough, asthenia, fatigue, hypertension, backpain, and binding anti-drug antibodies. No AE was found to positively correlate with exposure to the HMW species in the Phase 3 clinical study. Binding anti-drug antibodies were assessed for 2 patients and no unusual associations were observed between binding anti-drug antibodies and HMW species exposure. Accordingly, it was contemplated that HMW species can be tolerated in eculizumab pharmaceutical compositions that remain clinically suitable. For example, it is contemplated that eculizumab suitable for medical use may comprise 1.64% HMW species in a 900 mg dose, and may comprise 4.93% HMW species in a 300 mg dose

### Potency of Forced Degradation Samples Comprising HMW Species

In an additional set of experiments, eculizumab drug substance and drug product were subjected to forced degradation for 14 days at 50° C., which resulted in the formation of HMW species. After forced degradation, the drug substance (DS) comprised 7.0% HMW species as measured by SE-UHPLC (compared to 0.3% at Day 0, prior to forced degradation), and the drug product (DP) comprised 3.0% HMW species (compared to 0.3% at Day 0, prior to forced degradation). The forced degradation DS and DP comprising HMW species were assessed for relative potency of the eculizumab using the ELISA assay to measure inhibition of TCC formation as described in Example 1. As shown in Table 9, samples comprising 3.0% and 7.0% eculizumab retained relative potency comparable to control eculizumab samples. Thus, forced degradation samples comprising up to 7.0% HMW species of eculizumab retained a suitable potency profile.

TABLE 9

| | TimePoint at | HMW species | Relative Potency by Inhibition of TCC Formation |
|---|---|---|---|
| Sample | 50° C. (Day) | (%) | Assay (%) (% CV) |
| DS | 0 | 0.3 | 111 (7) |
| DS | 14 | 7.0 | 102 (2) |
| DP | 0 | 0.1 | 107 (4) |
| DP | 14 | 3.0 | 101 (2) |

### Example 4: Analysis of Methionine Oxidation Species

Methionine oxidation of eculizumab and reference product in clinical pharmaceutical compositions after different storage durations at 5° C. were determined by reduced peptide mapping. The methionine oxidized species exposure was calculated for lots suitable for medical use at the time of clinical administration (See International Patent App. No. PCT/US21/63641, published as WO 2022/132982). Linear regression profiles shown in Table 6 were applied to the eculizumab lots administered in a Phase 3 clinical trial. It was calculated that pharmaceutical compositions comprising eculizumab or reference product for clinical administration comprised up to 171.77 mg oxidized M253, 120.13 mg oxidized M359, and 121.39 mg oxidized M429, at the time of clinical administration. 171.77 mg oxidized M253 corresponds to 57.26% in a 300 mg dose, or 19.09% in a 900 mg dose of eculizumab. 120.13 mg oxidized M359 corresponds to 40.04% in a 300 mg dose, or 13.35% in a 900 mg dose of eculizumab. 121.39 mg oxidized M429 corresponds to 40.46% in a 300 mg dose, or 13.49% in a 900 mg dose of eculizumab. For clarity, patients were dosed in the Phase 3 study at 900 mg and the 300 mg dose was calculated based on the relative gravimetric amount of attribute exposure. AE analyzed included infection, headache, anemia, vaccination, pyrexia, cough, asthenia, fatigue, hypertension, backpain, and binding anti-drug antibodies. No AE was found to positively correlate with exposure to any of the methionine oxidized species in the Phase 3 clinical study. Binding anti-drug antibodies were assessed for 2 patients and no unusual associations were observed between binding anti-drug antibodies and methionine oxidation exposure.

Accordingly, it was contemplated that M253, M359, and M429 oxidized species can be tolerated in eculizumab pharmaceutical compositions that remain clinically suitable.

### Example 5: Analysis of Tryptophan Oxidation Species

Tryptophan oxidation of eculizumab and reference product in clinical pharmaceutical compositions after different storage durations at 5° C. were determined by reduced peptide mapping. The tryptophan oxidized species exposure was calculated for lots suitable for medical use at the time of clinical administration (See International Patent App. No. PCT/US21/63641, published as WO 2022/132982). Linear regression profiles shown in Table 6 were applied to eculizumab lots. Data were calculated for lots used in a Phase 3 clinical trial. It was calculated that pharmaceutical compositions comprising eculizumab or reference product for clinical administration comprised up to 113.29 mg oxidized W33, and 117.88 mg oxidized W107, at the time of clinical administration. 113.29 mg oxidized W33 corresponds to 37.76% in a 300 mg dose, or 12.59% in a 900 mg dose of eculizumab. 117.88 mg oxidized W107 corresponds to 39.29% in a 300 mg dose, or 13.10% in a 900 mg dose of eculizumab. For clarity, patients were dosed in the Phase 3 study at 900 mg and the 300 mg dose was calculated based on the relative gravimetric amount of attribute exposure. AE analyzed included infection, headache, anemia, vaccination, pyrexia, cough, asthenia, fatigue, hypertension, backpain, and anti-drug antibodies. No AE was found to positively correlate with exposure to any of the tryptophan oxidized species in the Phase 3 clinical study. Binding anti-drug antibodies were assessed for 2 patients and no unusual associations were observed between binding anti-drug antibodies and tryptophan oxidation exposure. Accordingly, it was contemplated that W33 and W107 oxidized species can be tolerated in eculizumab pharmaceutical compositions that remain clinically suitable.

The highest calculated of percent levels of oxidized M253, oxidized M359, and oxidized M429 (as shown in Example 4) and of oxidized W33 and oxidized W107 (as shown in Example 5) in eculizumab compositions and reference product compositions to which patients were exposed (in a 900 mg dose of eculizumab) and observed to be clinically suitable are summarized in Table 10, below:

TABLE 10

| | Eculizumab Composition: Highest | Reference Product: Highest |
|---|---|---|
| | Calculated Level of Patient Exposure | Calculated Level of Patient Exposure |
| | (based on a 900 mg dose) | (based on a 900 mg dose) |
| Oxidized Residue | (%) | (%) |
| M253 | 19.09 | 6.19 |
| M359 | 13.35 | 1.68 |
| M429 | 13.49 | 1.56 |
| W33 | 12.59 | 0.51 |
| W107 | 13.10 | 2.48 |

It will be appreciated that the values in Table 10 represent a 900 mg dose of eculizumab. For a 300 mg dose of eculizumab, based on the relative gravimetric amount of attribute exposure, it is contemplated that attribute levels at least three times (3×) as high as the percent levels of attributes for eculizumab shown in Table 10 can be clinically acceptable. For example, if a clinically acceptable 900 mg dose can comprise 13.10% oxidized W107, it is contemplated that a clinically acceptable 300 mg dose can comprise 39.30% oxidized W107.

It is noted that the reference product attribute exposure values in Table 10 are for the Phase 3 study reported in this example, and are determined using the methodology in the present example. These highest attribute exposure levels of the reference product have not been observed prior to the presently-described Phase 3 study and analysis. Peptide mapping had not been performed for the lots in the Phase 1 study described in Example 3.

It is contemplated that eculizumab compositions described herein may comprise the levels of any of the oxidized residues within ranges defined in Table 10. For example, the eculizumab compositions described herein may be greater than the highest calculated level of patient exposure for the reference product, and no more than the highest calculated level of patient exposure for the eculizumab composition as shown in Table 10.

### General

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the elements described herein, in all possible variations thereof, is encompassed by the disclosure unless otherwise indicated herein or otherwise clearly contradicted by context.

What is claimed:

1. A pharmaceutical composition comprising:
an anti-C5 antibody comprising:
a heavy chain comprising a CDRH1, CDRH2, and CDRH3, wherein the amino acid sequence of the CDRH1, CDRH2 and CDRH3 is SEQ ID NO: 1, 2 and 3, respectively, or SEQS ID NO: 4, 5, and 3, respectively; and
a light chain comprising a CDRL1, CDRL2, and CDRL3, wherein the amino acid sequence of the CDRL1, CDRL2, and CDRL3 is SEQ ID NO: 12, 13 and 14, respectively,
wherein the heavy chain comprises:
(a) M253 (EU numbering), wherein greater than 6% and no more than 20% of the anti-C5 antibody heavy chains of the composition comprise oxidized M253; and/or
(b) M359 (EU numbering), wherein greater than 2% and no more than 14% of the anti-C5 antibody heavy chains of the composition comprise oxidized M359; and/or
(c) M429 (EU numbering), wherein greater than 2% and no more than 14% of the anti-C5 antibody heavy chains of the composition comprise oxidized M429; and/or
(d) W33 (EU numbering), wherein greater than 1% and no more than 13% of the anti-C5 antibody heavy chains of the composition comprise oxidized W33; and/or
(e) W107 (EU numbering), wherein greater than 4% and no more than 14% of the anti-C5 antibody heavy chains of the composition comprise oxidized W107.

2. The pharmaceutical composition of claim 1, wherein the heavy chain comprises:
(a) M253 (EU numbering), wherein greater than 7% and no more than 20% of the anti-C5 antibody heavy chains of the composition comprise oxidized M253; and/or
(b) M359 (EU numbering), wherein greater than 5% and no more than 14% of the anti-C5 antibody heavy chains of the composition comprise oxidized M359; and/or
(c) M429 (EU numbering), wherein greater than 5% and no more than 14% of the anti-C5 antibody heavy chains of the composition comprise oxidized M429; and/or
(d) W33 (EU numbering), wherein greater than 2% and no more than 13% of the anti-C5 antibody heavy chains of the composition comprise oxidized W33; and/or
(e) W107 (EU numbering), wherein greater than 5% and no more than 14% of the anti-C5 antibody heavy chains of the composition comprise oxidized W107.

3.-6. (canceled)

7. The pharmaceutical composition of claim 1, wherein the heavy chain comprises:
(a) M253 (EU numbering), wherein greater than 7% and no more than 18% of the anti-C5 antibody heavy chains of the composition comprise oxidized M253.

8. The pharmaceutical composition of claim 1, wherein the heavy chain comprises:
(c) M429 (EU numbering), wherein greater than 4% and no more than 6% of the anti-C5 antibody heavy chains of the composition comprise oxidized M429.

9. The pharmaceutical composition of claim 1, wherein the heavy chain comprises:
(e) W107 (EU numbering), wherein greater than 6% and no more than 11% of the anti-C5 antibody heavy chains of the composition comprise oxidized W107.

10. (canceled)

11. (canceled)

12. The pharmaceutical composition of claim 1, wherein the anti-C5 antibody is authorized for administration to a human subject by a government regulatory agency.

13. (canceled)

14. The pharmaceutical composition of claim 1, wherein the percent oxidized amino acid residue is as determined by reduced peptide mapping comprising:
denaturing the anti-C5 antibody at a concentration of 1 mg/mL in 7.5M guanidine HCl, 0.25M Tris HCl, 2 mM EDTA pH 7.5;
reducing the denatured anti-C5 antibody in 9 mM Dithiothreitol (DTT) at 27° C.;
alkylating the denatured anti-C5 antibody in 16 mM Iodoacetic Acid (IAA) at 27 desalting the alkylated, denatured anti-C5 antibody;
digesting 1 mg/mL of the desalted anti-C5 antibody at pH 7.5 with trypsin at 0.08 mg/mL at 37° C., thereby producing peptides;
quenching the digest in 0.5% Trifluoroacetic Acid (TFA);
separating the peptides by reverse phase high performance liquid chromatography (RP-HPLC) on column comprising C4 chemistry, the separating comprising a mobile phase A of 0.1% TFA in water and mobile phase B of 0.1% TFA in acetonitrile (ACN) on a gradient comprising from 0.5% to 45% mobile phase B from 3 to 93 minutes at column temperature of 50° C., a sample tray temperature of 8° C., an injection volume of 50 μL, and a flow rate of 200 μL/minute; and
detecting the separated peptides by on-line mass spectrometry (MS) and MS/MS,
wherein (%) methionine oxidation is a frequency of the sum of all types of oxidation detected on the same methionine (M) residue; and wherein (%) tryptophan oxidation is a frequency of the sum of all types of oxidation detected on the same tryptophan (W) residue.

15.-16. (canceled)

17. The pharmaceutical composition of claim 1, comprising at least 1.7%, and no more than 5.0% HMW species as determined by SE-UHPLC.

18.-19. (canceled)

20. A pharmaceutical composition comprising:
an anti-C5 antibody comprising:
a heavy chain comprising a CDRH1, CDRH2, and CDRH3, wherein the amino acid sequence of the CDRH1, CDRH2 and CDRH3 is SEQ ID NO: 1, 2 and 3, respectively, or SEQS ID NO: 4, 5, and 3, respectively; and
a light chain comprising a CDRL1, CDRL2, and CDRL3, wherein the amino acid sequence of the CDRL1, CDRL2, and CDRL3 is SEQ ID NO: 12, 13 and 14, respectively,
wherein the pharmaceutical composition comprises no more than 5.0% HMW species anti-C5 antibody as determined by SE-UHPLC.

21. The pharmaceutical composition of claim 20, comprising greater than 1.5%, and no more than 5.0% HMW species as determined by SE-UPLC.

22. The pharmaceutical composition of claim 20, wherein the anti-C5 antibody has a relative potency of at least 80% compared to a reference anti-C5 antibody.

23.-27. (canceled)

28. The pharmaceutical composition of claim 1, wherein the anti-C5 antibody comprises a heavy chain variable region having the amino acid sequence of SEQ ID NO: 6 or 7 and a light chain variable region having the amino acid sequence of SEQ ID NO: 15.

29. (canceled)

30. The pharmaceutical composition of claim 1, wherein the anti-C5 antibody comprises a heavy chain a having the amino acid sequence of SEQ ID NO: 10 or 11 and a light chain having the amino acid sequence of SEQ ID NO: 17.

31.-33. (canceled)

34. The pharmaceutical composition of claim 1, further comprising a buffer comprising acetate.

35.-37. (canceled)

38. The pharmaceutical composition of claim 1, further comprising a chelating agent, wherein the chelating agent comprises ethylenediaminetetraacetic acid (EDTA) at a concentration of about 0.01 mM to about 0.05 mM.

39.-43. (canceled)

44. A method comprising administering the pharmaceutical composition of claim 1 to a subject in need of treatment by the anti-C5 antibody.

45. The method of claim 44, wherein the subject has myasthenia gravis, paroxysmal nocturnal hemoglobinuria, neuromyelitis optica spectrum disorder, or atypical hemolytic uremic syndrome.

46. The method of claim 44, wherein the anti-C5 antibody is administered to the subject at a dose of 300 mg-1200 mg or 600 mg-1200 mg, such as 300 mg, 600 mg, 900 mg, or 1200 mg.

47. (canceled)

48. A method of manufacturing the pharmaceutical composition of claim 1, comprising:
providing a composition comprising the anti-C5 antibody;
determining percentage of heavy chains of the anti-C5 antibody of the composition that comprise:
(a) M253 (EU numbering) oxidation
(b) M359 (EU numbering) oxidation,
(c) M429 (EU numbering) oxidation,
(d) W33 (EU numbering) oxidation; and/or
(e) W107 (EU numbering) oxidation; and
manufacturing the composition for pharmaceutical use if the heavy chain comprises:
(a) M253 (EU numbering), wherein greater than 6% and no more than 20% of the anti-C5 antibody heavy chains of the composition comprise oxidized M253; and/or
(b) M359 (EU numbering), wherein greater than 2% and no more than 14% of the anti-C5 antibody heavy chains of the composition comprise oxidized M359; and/or
(c) M429 (EU numbering), wherein greater than 2% and no more than 14% of the anti-C5 antibody heavy chains of the composition comprise oxidized M429; and/or
(d) W33 (EU numbering), wherein greater than 1% and no more than 13% of the anti-C5 antibody heavy chains of the composition comprise oxidized W33; and/or
(e) W107 (EU numbering), wherein greater than 4% and no more than 14% of the anti-C5 antibody heavy chains of the composition comprise oxidized W107.

49.-56. (canceled)

57. The method of claim 48, wherein the composition is manufactured for pharmaceutical use if the composition comprises no more than 5.0% HMW species of anti-C5 antibody as determined by SE-UHPLC.

58.-60. (canceled)


SEQUENCE LISTING (17 sequences)

SEQ ID NO: 1 - protein, 10 aa, synthetic construct
  REGION 1..10: note = DOE Sequence
GYIFSNYWIQ

SEQ ID NO: 2 - protein, 17 aa, synthetic construct
  REGION 1..17: note = DOE Sequence
EILPGSGSTEYTENFKD

SEQ ID NO: 3 - protein, 13 aa, synthetic construct
  REGION 1..13: note = DOE Sequence
YFFGSSPNWYFDV

SEQ ID NO: 4 - protein, 10 aa, synthetic construct
  REGION 1..10: note = DOE Sequence
GHIFSNYWIQ

SEQ ID NO: 5 - protein, 17 aa, synthetic construct
  REGION 1..17: note = DOE Sequence
EILPGSGHTEYTENFKD

SEQ ID NO: 6 - protein, 122 aa, synthetic construct [sequence omitted]

SEQ ID NO: 7 - protein, 122 aa, synthetic construct [sequence omitted]

SEQ ID NO: 8 - protein, 326 aa, synthetic construct
  REGION 1..326: note = DOE Sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 326 aa, synthetic construct
  REGION 1..326: note = DOE Sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 10 - protein, 448 aa, synthetic construct
  REGION 1..448: note = DOE Sequence
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 448 aa, synthetic construct
  REGION 1..448: note = DOE Sequence
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 12 - protein, 11 aa, synthetic construct
  REGION 1..11: note = DOE Sequence
GASENIYGALN

SEQ ID NO: 13 - protein, 7 aa, synthetic construct
  REGION 1..7: note = DOE Sequence
GATNLAD

SEQ ID NO: 14 - protein, 9 aa, synthetic construct
  REGION 1..9: note = DOE Sequence
QNVLNTPLT

SEQ ID NO: 15 - protein, 107 aa, synthetic construct [sequence omitted]

SEQ ID NO: 16 - protein, 107 aa, synthetic construct [sequence omitted]

SEQ ID NO: 17 - protein, 214 aa, synthetic construct [sequence omitted]